(19) United States
(12) Patent Application Publication
(10) Pub. No.: US 2024/0420848 A1
(43) Pub. Date: Dec. 19, 2024

(54) SYSTEMS AND METHODS FOR USING MACHINE-LEARNING TO PREDICT PATIENT HEALTH CHARACTERISTICS

(71) Applicant: Health Outlook Corp., New York, NY (US)

(72) Inventors: Howard W. SAMS, Rye, NY (US); Leena DAS-YOUNG, Naples, FL (US); David H. PERLMAN, New York, NY (US); Adam BRZOZOWSKI, Mansfield, MA (US); Elaine E. THOMPSON, Silver Spring, MD (US)

(21) Appl. No.: 18/742,803

(22) Filed: Jun. 13, 2024

Related U.S. Application Data
(60) Provisional application No. 63/659,177, filed on Jun. 12, 2024.
(60) Provisional application No. 63/555,730, filed on Feb. 20, 2024.
(60) Provisional application No. 63/508,582, filed on Jun. 16, 2023.

(51) Int. Cl. G16H 50/30; G16H 10/60

(52) U.S. Cl. CPC G16H 50/30; G16H 10/60

(57) ABSTRACT

Methods for predicting health characteristic may include: obtaining patient medical information that includes protein data; a machine-learning model based on concordance with the characteristic, the patient medical information, a time-frame for prediction, or in the protein data. Different models may have been trained using different groupings of training patient data, trained to predict different characteristics, trained or evaluated using different biomarkers, trained using proteins, protein groups, or protein categories listed in Tables M through R, or evaluated for different time-frames. The selected model may be used to generate the prediction using the patient medical information. A model may be generated by pre-processing medical information, generating an ensemble of models; determining a sensitivity of variables of the medical information to identify a subset of variables; and training a machine-learning model using the subset.

### RELATED APPLICATIONS

This disclosure is a non-provisional application of, and claims the benefit of priority to U.S. Provisional Application No. 63/659,177, filed Jun. 12, 2024, to U.S. Provisional Application No. 63/555,730, filed Feb. 20, 2024, and to U.S. Provisional Application No. 63/508,582, filed Jun. 16, 2023, the disclosures of which are incorporated herein in their entireties.

### TECHNICAL FIELD

Various embodiments of this disclosure relate generally to machine-learning-based techniques for predicting patient health characteristics, and, more particularly, to systems and methods for preparing and using health study data with an ensemble machine-learning system to make predictions on patient health characteristics.

### BACKGROUND

Current methods for diagnosis and outcome prediction tend to rely on either (i) one or two markers, or (ii) on indication of obvious disease. While individual biomarkers are widely used in medical diagnosis, they are primarily used for diagnosis, rather than being predictive. Moreover, stratifying risk based on individual measurements and standard lab panels is difficult, and may interfere with patient care. Risk factors such as risk scores generated using statistical patient data may be predictive of certain aspects of patient health, but they are largely behavioral and might not provide sufficient insight as to whether underlying biological mechanisms involved in the development of various disease are active. Such conventional risk factors or statistical analyses may not provide insights in a concrete, actionable time frame. Many people only learn about their disease or condition after onset of symptoms, e.g., when they are admitted to the emergency room, e.g., with a heart attack or other severe condition.

This disclosure is directed to addressing one or more challenge such as those referenced above. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for machine-learning-based techniques for predicting patient health characteristics, and, more particularly, to systems and methods for preparing and using health study data with an ensemble machine-learning system to make predictions on patient health characteristics.

Early warning panels of biomarkers and associated multivariate artificial intelligence/machine learning (“AI/ML”) models facilitate prediction of which people are at elevated risk of a health event. In an example, predictions generated with such a model may be usable to identify people that are at risk for one or more conditions in the future. Such identification enables monitoring high risk individuals to ensure a rapid response, and applying interventions for moderate-risk people, e.g., lifestyle modifications that may inhibit a dangerous health event. Additionally, more accurate predictions of various conditions, both short-term and long-term, which utilize such advances may help various healthcare stakeholders plan for capacity and manage risk.

In one aspect, an exemplary embodiment of a computer-implemented method for generating a prediction of one or more patient health characteristic may include: obtaining patient medical information that includes protein data; automatically selecting one or more machine-learning model from amongst a bank of trained machine-learning models based on a concordance of the one or more machine-learning model with one or more of the one or more patient health characteristic, patient data in the patient medical information, one or more time-frame for the prediction, or proteins included in the protein data, wherein different machine learning models in the bank were one or more of trained using different stratifications/groupings/classifications of training patient data, trained to predict different patient health characteristics, trained or evaluated using different sets of proteins, or evaluated for different time-frames; and
generating the prediction of the one or more patient health characteristic by evaluating the patient medical information using the one or more selected machine learning model.

In another aspect, an exemplary embodiment of a computer-implemented method of generating a machine-learning model for predicting a patient health characteristic may include: obtaining longitudinal medical information; performing at least one pre-processing operation on the longitudinal medical information; generating an ensemble of models that relate the longitudinal medical information to a patient health characteristic of interest; applying the ensemble of models to at least a portion of the longitudinal medical information; determining one or more of an impact or importance of variables of the longitudinal medical information by evaluating results from applying the ensemble of models to the longitudinal medical information; identifying a subset of the variables based on the determined one or more of the impact or importance; training the machine-learning model using the subset of the variables of the longitudinal medical information.

In a further aspect, an exemplary embodiment of a computer-implemented method for generating a prediction of one or more patient health characteristic may include: obtaining patient medical information that includes protein data; generating the prediction of the patient health characteristic by evaluating the patient medical information with a trained machine-learning model, wherein the machine-learning model has been trained using one or more of a plurality of the proteins, a plurality of protein groups, or a plurality of protein categories listed in Tables M through R.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary environment for one or more of training or using at least one machine-learning model to generate predictions for patient health characteristics, according to one or more embodiments.

FIG. 2 depicts a flowchart of an exemplary method of generating and/or training a machine-learning model to generate predictions for patient health characteristics, according to one or more embodiments.

FIG. 3 depicts a flowchart of another exemplary method of generating and/or training a machine-learning model to generate predictions for patient health characteristics, according to one or more embodiments.

FIG. 4A depicts a graph of an experimental time-dependent concordance of a machine-learning model trained according to FIG. 2.

FIG. 4B depicts an exemplary illustration of a visual representation of output from a machine-learning model, according to one or more embodiments.

FIG. 4C depicts an illustration of exemplary risk assessments output from a machine-learning model, according to one or more embodiments.

FIG. 4D depicts an exemplary chart illustrating a patient's predicted longevity over time output from a machine-learning model, according to one or more embodiments.

FIG. 4E depicts an exemplary demographic assessment for a patient output from a machine-learning model, according to one or more embodiments.

FIG. 4F depicts an exemplary cumulative hazard function for a plurality of patients output from a machine-learning model, according to one or more embodiments.

FIG. 5 depicts a flowchart of a further exemplary method of generating and/or training a machine-learning model to generate predictions for patient health characteristics, according to one or more embodiments.

FIG. 6 depicts a flowchart of an exemplary method for generating one or more prediction for one or more patient health characteristic.

FIG. 7 depicts a flowchart of another exemplary method for generating one or more prediction for one or more patient health characteristic.

FIGS. 8A-8B depict Table M of protein biomarker data.

FIGS. 9A-9D depict Table N of protein biomarker data.

FIGS. 10A-10C depict Table O of protein biomarker data.

FIGS. 11A-11D depict Table P of protein biomarker data.

FIGS. 12A-12D depict Table Q of protein biomarker data.

FIGS. 13A-13D depict Table R of protein biomarker data.

### DETAILED DESCRIPTION OF EMBODIMENTS

According to certain aspects of the disclosure, methods and systems are disclosed for generating predictions of patient medical characteristics, e.g., longevity, risk of disease or the like. Medical information, such as protein biomarkers, are commonly used diagnostically. However, conventional techniques may not be suitable for considering medical information to make predictions. For example, conventional techniques may not be sufficiently accurate, patient-specific, or actionable. Accordingly, improvements in technology relating to understanding and drawing insights from medical information are needed.

As will be discussed in more detail below, in various embodiments, systems and methods are described for using machine learning to generate predictions for patient medical characteristics. By training a machine-learning model, e.g., via supervised or semi-supervised learning, to learn associations for medical information sourced over time, the trained machine-learning model may be usable to generate predictions based on current and historical medical information of a patient.

Reference to any particular activity is provided in this disclosure only for convenience and not intended to limit the disclosure. A person of ordinary skill in the art would recognize that the concepts underlying the disclosed devices and methods may be utilized in any suitable activity.

The terminology used herein may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. The description herein is exemplary and explanatory only and are not restrictive of the features, as may be understood by one of ordinary skill in the art.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (GBM), deep learning, and/or a deep neural network. Supervised learning may include providing training data and labels corresponding to the training data, e.g., as ground truth.

Machine learning techniques adapted to predicting patient health characteristics may include one or more aspects according to this disclosure, e.g., a particular selection of training data, a particular training process for the machine-learning model, operation of a particular device suitable for use with the trained machine-learning model, operation of the machine-learning model in conjunction with particular data, modification of such particular data by the machine-learning model, etc., and/or other aspects that may be apparent to one of ordinary skill in the art based on this disclosure.

In an example, a patient may desire or benefit from predictions relating to one or more health condition or risk factor. Predictions can be a powerful tool in long-term, sustainable, and optimally interventional care. For instance, lifestyle changes, medication, or the like, if enacted early such as before symptoms develop may reduce, inhibit, or prevent a more acute condition. In comparison, diagnostic approaches generally do not enable intervention until a condition progresses to the point where the patient is compelled to seek care. Statistical analysis has been used to identify risk factors that are predictive of certain aspects of patient health. However, risk factors are generally behavioral and might not provide sufficient insight as to whether underlying biological mechanisms involved in the development of various disease are active. Such conventional risk factors or statistical analyses may not provide insights in a concrete, actionable time frame.

One confounder in analysis of health conditions is that the number of conditions and variables available for analysis is myriad. While statistical analysis has been used to show correlations between some factors and some conditions, the non-trivial interrelation of various patient characteristics results in an imperfect-at-best understanding of the underlying mechanism of any condition or symptom experienced by a patient. This understanding is stretched even more thinly when applied predictively. Conventional techniques may not be able to accurately predict long-term effects of seemingly benign patient characteristics, and thus may be unable to accurately assess patient risk.

In an exemplary use case a provider may obtain medical information for a patient, e.g., one or more of demographic information, medical history, lifestyle history, or the like. The provider may also obtain, for example, one or more biomarkers. For example, the provider may take a measurement (e.g., pulse, blood pressure, etc.) collect a sample (e.g., blood, tissue, etc.), perform imaging (x-ray, magnetic resonance, etc.), and use such information to determine and/or quantify one or more characteristics of the patient. Conventionally, such medical information is useful diagnostically, e.g., to determine or describe a present characteristic of the patient, but has more limited utility when used predictively. As noted above, expertise of an experienced provider and/or statistical correlations may be used to form general predictions. However, more patient-specific predictions, as well as predictions for a variety of time-frames, may be beneficial. In one aspect, patient medical information, and in particular results from a protein test for a particular set of proteins, may be fed into one or more trained machine-learning models to generate one or more predictions for a characteristic of the patient.

In one aspect, one or more models may be selected on a patient-specific basis from a bank of models. For instance, different models may be trained using training data sourced from patients of different demographics, having different characteristics or the like. In an example, one or more models may be selected based on concordance between characteristics of the patient and characteristics of a patient population used to train the one or more models.

In one aspect, one or more models may be selected based on a desired timeframe for prediction. For example, in an embodiment, different models may exhibit different accuracy and/or different concordance with the patient for different time-frames. For instance, one model may have a higher accuracy for a 10-15 year prediction than another model, but the other model may have a higher accuracy for a 15-20 year prediction.

In one aspect, predictions for multiple time-frames may be generated using medical information from a single acquisition. For example, a single blood test of a patient to acquire protein biomarkers may be used, e.g., with different models for different timeframes, to generate predictions across multiple time-frames.

In one aspect, different providers and/or protein tests may include different sets of proteins. One or more models may be selected based on the included sets of proteins. For instance, an optimal model or models may be selected based on the particular proteins included in the set. In an example, a first model may include proteins A and B as input, and a second model may include proteins A, B, and C as input. If a protein panel for the patient only includes proteins A and B, then in some embodiments, the second model may be selected due to protein C being missing for the first model. In some embodiments, models may be configured to accept null values or default values for missing inputs. Thus, in an example, an accuracy and/or concordance between the patient and the first and second models may be performed given the proteins available for the patient. In some instances, the first model may be more optimal for the patient, even with a null value or default value for protein C. In another embodiment, the protein data for the patient may include proteins A, B, C, and D, and either of the first or second model may be selected based on concordance or accuracy given the available patient data.

In one aspect, different models may be configured, e.g., via their training, architecture, or the like, to make predictions for different patient characteristics. For instance, different models may be used to generate predications regarding, for example, likelihood of the patient to exhibit (e.g., within a given time-frame for the prediction), longevity or mortality (all-cause-mortality), kidney disease, heart failure, atrial fibrillation, depression, cancers (e.g., of the lungs, stomach, liver, esophagus, breast, thyroid, colorectal, etc., Alzheimer's or dementia, chronic obstructive pulmonary disease, stroke, neurodegenerative disease, peripheral arterial disease, arthritis, hypertension, diabetes, etc. As discussed in further detail below, such models may be used to generate quantifiable patient-specific predictions for such characteristics across one or more discrete time-frames.

In one aspect, machine-learning provides for consideration of the correlation and interrelation of a wide variety of factors not conventionally possible through professional expertise or statistical analysis. Consideration of patient medical data enriched with additional biomarkers that might not be conventionally considered or considered in combination may enable more accurate and more patient-specific predictions. In an example, additional data such as data from wearables (fitness trackers, step meters, altimeters, accelerometers, etc.), prescription data, life and medical events and the like, which may not be conventionally considered when assessing patient characteristics may be quantified and/or fed into a model as input.

In one aspect, patient data acquired over time may be used to update, tune, and/or retrain one or more models. For example, a model configured to generate a prediction in a 25 year time-frame may be refined over time as patients age, such that data is available at a current time and at a time corresponding to the prediction time-frame (e.g., 25 years ago).

In one aspect, an exemplary embodiment utilizes one or more sets of biomarkers, e.g., from one or more of the examples discussed elsewhere, and one or more matching AI/ML models. In an example, data from a new patient may be acquired, e.g., including a set of proteins. For example, a Luminex assay may be used to concurrently measure a set of proteins. In various embodiments, protein biomarkers may be measured qualitatively and/or quantitatively, e.g., from blood, serum, plasma, urine, saliva, or other bodily fluids either individually, multiplexed, or through proteomics using analytic techniques including but not limited to immunoassays, immunostaining, enzyme activity assays, affinity binding techniques, flow cytometry, mass spectrometry, aptamers, protein microarrays, or any other suitable technique. A collection of AI/ML predictive models may be used to assess both the short-term and long-term medical outlook for the patient, and/or to identify undiagnosed health conditions of the patient.

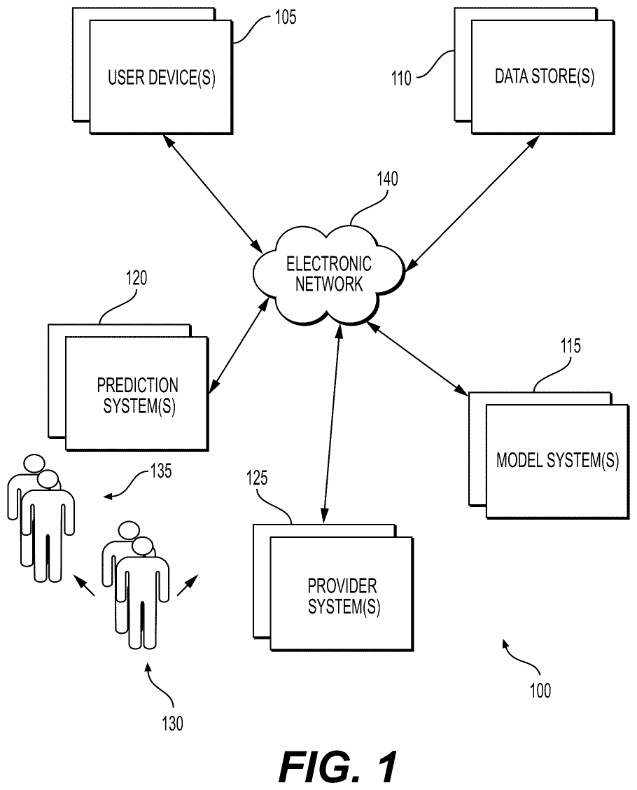

FIG. 1 illustrates an exemplary environment 100 for evaluating patient medical information, e.g., to generate a prediction for one or more patient medical characteristics in one or more time-frames. As illustrated in FIG. 1, the environment 100 may include one or more user device(s) 105, one or more data store(s) 110, one or more model system(s) 115, one or more prediction system(s) 120, and one or more provider system(s) 125. The provider system(s) 125 may be associated with a provider 130, e.g., a medical practitioner, a hospital, a laboratory, or an agent or entity associated therewith. The provider 130 may be associated with a patient 135, e.g., a person who may desire or benefit from predictive information regarding one or more medical characteristics of the patient 135. The systems and devices of the environment 100 may communicate in any arrangement, e.g., via an electronic network 140.

In an exemplary embodiment, a user, e.g., a patient 135, a provider 130, etc., may utilize the user device 105 to perform one or more activities as discussed here, and/or to communicate with another device in the environment 100. In some embodiments, the components of the environment 100 are associated with a common entity, e.g., the provider 130, or the like. In some embodiments, one or more of the components of the environment 100 are associated with a different entity than another. As will be discussed herein, systems and/or devices of the environment 100 may communicate in order to one or more of generate, train, or use a machine-learning model to predict patient health characteristics, among other activities.

The user device 105 may be a computer system such as, for example, a desktop computer, a mobile device, a tablet, etc. In some embodiments, the user device 105 may include one or more electronic application(s), e.g., a program, plugin, browser extension, etc., installed on a memory of the user device 105. In some embodiments, the electronic application(s) may be associated with one or more of the other components in the environment 100. For example, the electronic application(s) may include one or more of system control software, system monitoring software, software development tools, a patient portal, a provider portal, prediction software, an Electronic Medical Records (EMR) portal, etc.

The data store 110 may include a computer system, such as a server system, an EMR system, or the like, and may include computer-readable memory such as a hard drive, flash drive, disk, etc. In some embodiments, such the data store 110 includes and/or interacts with an application programming interface for exchanging data to other systems, e.g., one or more of the other components of the environment 100. The data store 110 may include and/or act as a repository or source for patient health data, model data, etc. For example, the data store 110 may store patient medical information such as demographic data, historical patient characteristics, biomarkers, etc. In an example, the data store 110 may store patient data from different points in time, e.g., longitudinal data. In some embodiments, the data store 110 may include data from or have access to one or more patient information resources, e.g., historical patient study data or the like.

The provider system 125 may include a computer system configured to enable a provider 130 to input patient medical information, e.g., resulting from an examination, laboratory test, or the like. In some embodiments, the provider system 125 may be configured to communicate with other elements of the environment 100, e.g., to provide data, to request a prediction, to output results of a generated prediction, or the like.

In various embodiments, the electronic network 140 may, for example, be a wide area network (“WAN”), a local area network (“LAN”), personal area network (“PAN”), or the like. In some embodiments, electronic network 140 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

The model system 115 may include a computer system that stores one or more models and/or one or more algorithms, tools, or the like for generating, training, and/or using such models. In an example, the model system 115 may include a machine-learning model and/or instructions associated with the machine-learning model, e.g., instructions for generating a machine-learning model, training the machine-learning model, using the machine-learning model etc. Generally, a machine-learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, one or more algorithms or tools may be used to pre-process data in order to generate training data, as discussed in more detail below. In some embodiments, a portion of the training data may be withheld during training and/or used to validate the trained machine-learning model, e.g., compare the output of the trained model with the ground truth for that portion of the training data to evaluate an accuracy of the trained model. The training of the machine-learning model may be configured to cause the machine-learning model to learn associations between one or more variables of patient health data and one or more patient health characteristics and/or outcomes, such that the trained machine-learning model is configured to determine an output prediction of a health characteristic or outcome in response to the input patient health data based on the learned associations.

In various embodiments, the model system 115 may store one or more of a linear model, a least-square or partial least-square regression model, a recursive partitioning model such as a Cox model, a gradient boosted Cox model, a Cox Random Forest Model, etc., a neural network, e.g., a deep neural network, a support vector machine, a Naïve Bayes model, etc. Moreover it should be understood that the foregoing are examples only. Any suitable type of model, e.g., machine learning model or combination of machine learning models may be used. Operations conducted by one model in some embodiments may be distributed amongst a plurality of models in other embodiments, or vice versa.

The prediction system 120 may include a computer system, a portal, an API, or the like, and may be configured to operate, access, or work in conjunction with other elements of the environment 100, e.g., to generate predictions of patient medical characteristics. In an exemplary use case, the prediction system may obtain medical information of a new patient, e.g., from the provider system 125 and/or the data store 110, may access one or more trained models that pertain to the new patient, e.g., on the model system 115, and generate one or more patient medical characteristic prediction for the new patient. In some embodiments, the prediction system 120 may be configured to pre-process the obtained medical information, e.g., to interpolate missing data, set default or null values for missing data, etc.

Although depicted as separate components in FIG. 1, it should be understood that a component or portion of a component in the environment 100 may, in some embodiments, be integrated with or incorporated into one or more other components. For example, a portion of the user device 105 may be integrated into the prediction system 120, the provider system 125, or the like. In another example, the model system 115 may be integrated the prediction system 120, and/or the data store 110 may be integrated into the model system 115, etc. In some embodiments, operations or aspects of one or more of the components discussed above may be distributed amongst one or more other components. Any suitable arrangement or integration of the various systems and devices of the environment 100 may be used.

In the following methods, various acts may be described as performed or executed by a component from FIG. 1, such as the model system 115, the prediction system 120, the user device 105, etc. However, it should be understood that in various embodiments, various components of the environment 100 discussed above may execute instructions or perform acts including the acts discussed below. An act performed by a device may be considered to be performed by a processor, actuator, or the like associated with that device. Further, it should be understood that in various embodiments, various steps may be added, omitted, or rearranged in any suitable manner.

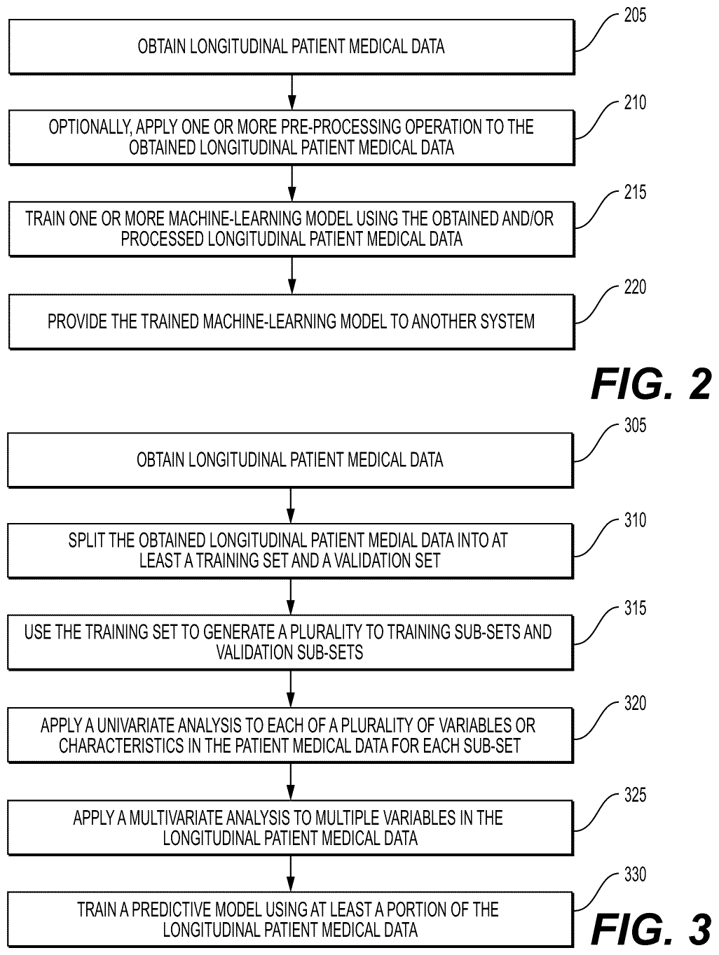

FIG. 2 depicts an exemplary methodology for developing, e.g., training, a prediction model for generating one or more patient medical characteristics predictions, such as in one or more of the examples above. At step 205 longitudinal patient medial data may be obtained. In an example, data may be accessed from a long-term study in which patient medical data including protein biomarker data was collected. In an example, one or more studies may be selected based on, for example, length of study, retention rate, and collected characteristics.

In some embodiments, different studies, pools of patients, or the like are accessed for training different models. For example, a different data source may be selected when training a longevity model as compared to a model for predicting Alzheimer's, etc. In some embodiments, patient data from one or more studies is extracted based on the presence or lack of presence of a particular characteristic. For instance, when training a model for which a particular biomarker is relevant, patient data for which that particular biomarker was not collected may be excluded. In another instance, when training a model for a particular population segment (age, region, demographic, etc.), patient data not relevant to that segment may be excluded.

Collected characteristics may include, for example, one or more of heart health, an extensive number of risk factors or biomarkers, timing and severity of cardiovascular diseases, and/or eventually mortality and primary cause of death. Risk factors or biomarkers may include protein and/or biomarker data, outcome data, demographic and basic patient characteristic data, lab results data, vitals, and/or patient health status data. Outcome data may include patient years of survival after measurement of predetermined patient characteristic data, lab result data, vitals, and or patient health status data. Demographic and patient characteristic data may include age, sex, geographic location, ethnicity, blood pressure, smoking, body mass index (“BMI”), height/waist ratio, and/or heart rate. Lab result data may include patient HDL cholesterol, total cholesterol, asymmetric dimethylarginine, symmetric dimethylarginine, homocysteine, cysteine, triglycerides, L-arginine, blood glucose, cysteine, serum creatinine, folate, homocysteine, Vitamin B12, Vitamin B6, and/or other blood chemistry results. Vitals may include diastolic blood pressure systolic blood pressure, ventricular heart rate, BMI, waist/height ratio. Health status data may include lifestyle characteristics, whether the patient smokes, quantity of tobacco intake, history of stroke, insulin quantity, dilated left ventricular hypertrophy, whether the patient is on hypertension medication, is on lipid lowering medication, is on blood sugar medication, quantity of insulin taken per day, and/or is being treated for diabetes and/or heart disease. Protein biomarker data is discussed in further detail below with regard to Tables A-R.

Optionally, at step 210, one or more pre-processing operation may be applied to the obtained patient medical data. In one example, e.g., in an embodiment in which patient data is obtained from different studies or sources, and thus may not be in a standardized format, pre-processing may include a data standardization process.

In an experimental example, data obtained from a three-generational study included results with missing data, e.g., data collection instances where one or more characteristics of a patient were not collected or known. Conventionally, missing data of this type may be addressed via imputation, but such techniques may lead to lowered model accuracy. Thus, in various embodiments, one or more of variable and subject selection may be balanced to minimize imputation, but retain protein biomarkers, key risk factors, and as many subjects as possible.

In a further example, variables in the data may be evaluated to identify any that were not consistently measured during the study. For instance, not all biomarkers may have been collected early on in the study, or certain biomarkers may have been dropped or replaced with others. In an example, a predetermined threshold may be used to determine whether to retain variables. For instance, if a variable is missing from 1%, 5%, 10%, etc., or more of collected samples, that variable may be dropped.

In another example, variables may be dropped that are redundant of or correlated with one or more other variables, e.g., one or more variables that had a higher or more consistent representation. For instance, a low-density lipoprotein (“LDL”) variable with a number of missing values exceeding a threshold may be removed in favor of other measurements in a lipid profile that have less missing values and that can be linearly combined to determine LDL.

In another example, a threshold quantity of missing measurements may be used to evaluate the data, and any variables missing more measurements than the threshold may be eliminated. In an experimental example, a threshold value of 100 was determined empirically to balance having as many subjects as possible with complete data vs. as many variables as possible to evaluate for model development. In another example, a threshold value may be selected based on a difficulty involved in filling in missing data via imputation. As an illustrative example, a certain protein biomarker with missing values in 4% of the cohort was used in establishing a threshold of ˜4% of the cohort due to the difficulty in imputing a biomarker across that portion.

In another aspect, imputation and/or interpolation may be used to replace a threshold number of missing values. In one example, missing values about medication use may be set to zero/no. In another example, information from a later testing date may be used to impute missing information for the same characteristic at a different date, e.g., smoking at a later visit may be imputed to fill in missing data about smoking at another visit. In another example, missing values for a specific indicator or condition, e.g., the presence of dilated left ventricular heart failure, may be imputed to zero/no. In a further aspect, after performing techniques such as the above, any patient remaining that has missing values may be dropped from the training set. In an experimental example, techniques such as the above were applied to an initial cohort of 2,606 individuals to produce a 2,448 person training set.

As should be understood, one or more of the techniques above may result in a reduction of variables. In some embodiments, the remaining variables may be inspected for redundancies. When possible, quantitative data may be used in favor of categorical data. For example, this may include using kit variables for folate assays instead of L. casei; using units of insulin per day instead of presence/absence of insulin use, using cigarettes per day instead of a binary yes/no for smoking; using a waist/height ratio in favor of a waist measurement, etc.

In an illustrative example, after performing one or more pre-processing techniques, the remaining variables for the patients of the study in the training set may include, for example, outcome (e.g., survival for a survival study, presence of a particular disease or condition, etc.), demographic data, patient vitals, medical history (e.g., medications, past diagnoses or medically relevant events, medically relevant habits, etc.), protein biomarkers, results from labs or panels, or the like.

At step 215, the patient medical data, e.g., pre-processed patient medical data, may be used to train one or more machine-learning model. In one aspect of this disclosure, machine learning techniques such as gradient boosted Cox models and/or neural networks may be used to model the interrelationship between multiple variables. In one aspect, a gradient boosted model may be used. Such a model may exhibit a relatively rapid training speed compared to other models, which may facilitate iterative model development, higher parsimony, and (as observed experimentally after training) an improved reliance on protein biomarkers relative to other health characteristics in the patient medical data. However, it should be understood that any suitable type of model or combination of models may be used in various embodiments. Since the data set was relatively small compared to typically data sets used with AI/ML techniques, a smaller number of variables in the model may tend to reduce the degree of overtraining.

In some embodiments, multiple models may be trained as in intermediary step in order to identify optimal variables before using the identified variables to train a final model. Regularization for training of a gradient-boosted Cox forest may be used to optimize factors such as learning rate, subsampling, and/or a number of estimators in the model. In an exemplary model development process, different models were repeatedly trained with varied parameters and different numbers of estimators in order to identify parameters that provide the highest concordance without notable deterioration of the model or overtraining. As used herein, “concordance” as applied to longitudinal data may encompass a rate at which predictions based on earlier portions of longitudinal data align with actual values of later portions of the longitudinal data, and/or may encompass a rate at which predictions for patient medical data withheld from the training process so as to be used as validation data are accurate.

The identified parameters may then be used to train a further model. In an experimental example, the further model was trained on a stratified training set consisting of 64% of an obtained set of patient medical data, whereby the remaining 36% was used for validation. The trained final model was found to have a good concordance of 0.78% with the validation data.

In one aspect, after parameterization, e.g., as discussed above, one or more individual weight of each parameter on the output of the model may be determined, e.g., in order to reduce complexity of the further model. In the experimental example above, several parameters had low or zero value weights. Such determinations may be used to remove such parameters from the further model to generate (and then train) a refined model. Such removal may reduce the parameters needed from new patients, simplify or improve the training or re-training process on future models, etc.

At step 220, a trained model may be provided to another system, e.g., prediction system 120, or the like.

FIG. 3 depicts another exemplary methodology for developing, e.g., training, a prediction model for generating one or more patient medical characteristics predictions, such as in one or more of the examples above. The methodology below is discussed for predicting mortality/longevity. However, it should be understood that a similar methodology may be used for predicting presence of any suitable patient medical characteristic. At step 305 longitudinal patient medial data may be obtained.

At step 310, the obtained patient medical data may be split into a training set and a validation set.

At step 315, the training set may be used to generate a plurality of training sub-sets and validation sub-sets. In an example, generating the sub-sets may include a resampling process, such as stratified random splitting using one or more patient characteristics such as age, gender, the characteristic of interest (e.g., mortality), etc. Such a sampling process may facilitate preserving relative proportions of such characteristics across the sub-sets.

At step 320, univariate analysis may be applied to each of a plurality of variables or characteristics in the patient medical data for each sub-set. In an example, the training set may be resampled to obtain subsets sampled for mortality, gender, particular age ranges, etc. For each sub-set, a single variable, e.g., presence of Protein A, may be used to train a univariate model controlled for the characteristic of interest. For example, the variable for Protein A from a training sub-set limited to patients in an age range of 49-56 may be used to train a model, along with data regarding the characteristic of interest for the included patients, i.e., their mortality. The trained model may then be applied to the corresponding validation sub-set and then evaluated against the characteristic of interest for the patients in the validation sub-set in order to determine a relative impact/importance/sensitivity of that variable on the characteristic of interest. The relative impact of multiple variables may be used to generate a ranked list of variables. A sub-set of the ranked variables may be selected, e.g., based on one or more of a relatively high rank, experimental data, expertise, etc.

At step 325, multivariate analysis may be applied to multiple variables in the patient medical data. In an example, a relatively narrow segment of the population may be isolated, e.g., patients over 50 years of age. For a characteristic such as mortality, truncation bias may affect sample size for later periods of prediction. To offset such bias, a down-sampling process may be used. In an example specific to the generation of a mortality/longevity model, all subjects that died over the course of the study may be identified. An equal number of subjects that lived through the study may also be identified, e.g., via a random and/or constrained resampling process. The identified deceased and alive subjects may be combined into a multivariate training set. A similar down sampling process may be performed to generate multivariate training sets for other characteristics.

In some embodiments, not all of the variables in the patient medical data is incorporated into a multivariate training set. For example, in some embodiments, the sub-set of variables identified in step 320 may be used. In an experimental example for generating a mortality/longevity model, such a sub-set of variables may include: age, gender, smoking habits or history, statin use, cancer status, cardiovascular status, metabolic equivalent (MET), body-mass index (BMI), diabetes status or history, systolic or diastolic pressure, blood chemistry characteristics, protein biomarkers, etc.

A plurality of different models may then be used to evaluate the multivariate training set, e.g., one or more of a gradient boosted machine, a linear model (e.g., partial least squares discriminant analysis), a Lasso and Elastic-Net Regularized Generalized Linear Model (GLM-NET), a random forest model, a neural network (e.g., a deep neural network), a recursive partitioning model, a naïve Bayes model, a support vector machine, or any other suitable model or combinations thereof. Each model may be separately tuned based on one or more variables in order to find an optimal fit to the test data as evaluated by cross-validation. In an example, for each model tested in this manner, sensitivity may be evaluated against specificity, the results of which may be evaluated, e.g., integrated, in order to determine an overall performance metric for each model. The performance metric may be used to identify an optimal model type for a predictive model.

The models may also be evaluated for variable importance e.g., via plotting histograms or probability plots of each variable for each model. Any other suitable evaluating technique is also usable. The importance of the variables may be compared against the rank determined via univariate analysis above. In an example, a higher importance value and a lower rank may be indicative that a particular variable is relevant across a variety of models. Such relevant variables may thus be good candidates for use in the predictive model. In some embodiments, a plurality of relevant variables are identified.

At step 330 the predictive model is trained. For example, a model type, identified based on the performance metric from the multivariate analysis, may be trained, e.g., using the identified relevant variables only from the training set or a sub-set.

In one aspect, a model may be assessed for time-dependent concordance. For example, concordance of a particular model may be assessed against validation data at different time windows, e.g., over yearly segments, segments of 2.5 years, 5 years, 10 years, etc. The resulting concordance values may then be evaluated, e.g., to determine a time-dependent concordance of the model.

For instance, a model predicting survival or longevity such as in an all-cause mortality model may be more or less accurate over different windows of time. In an example, many of the patients included in the training set may have remained alive for 15-20 years after the most recent data was obtained. The model may have a higher concordance for a time range in which more sample data is available.

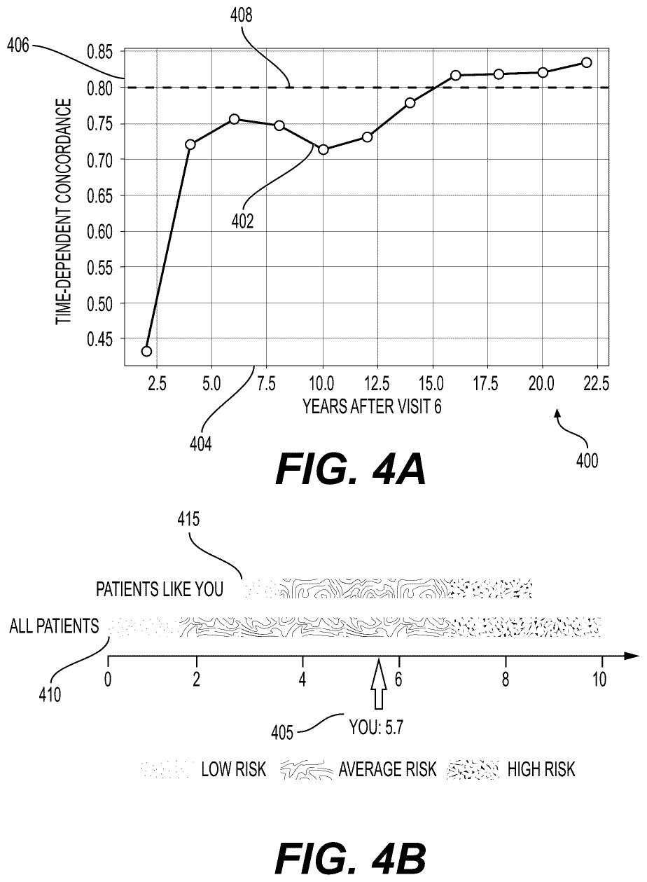

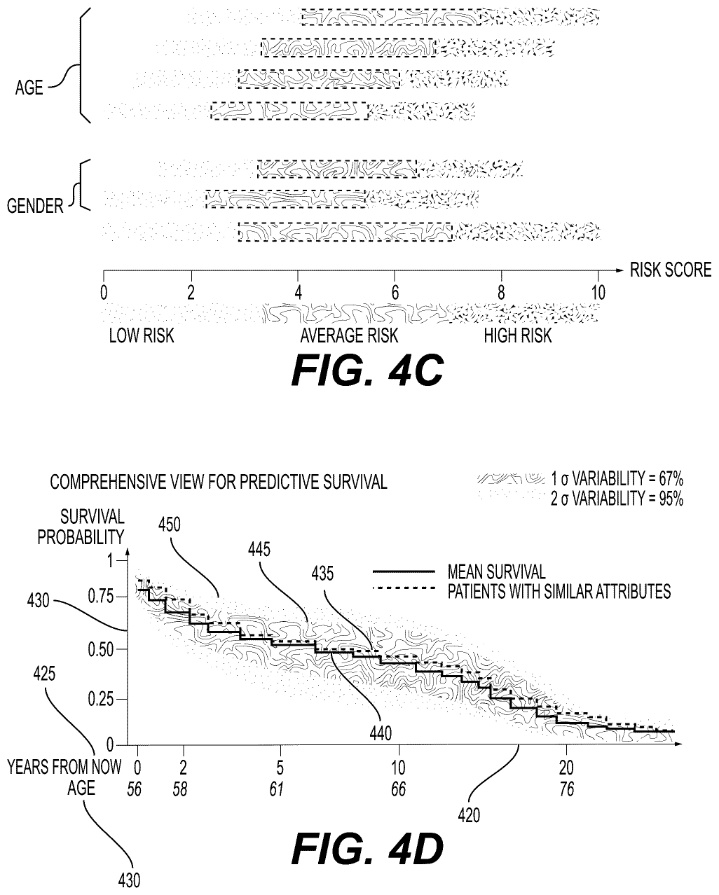

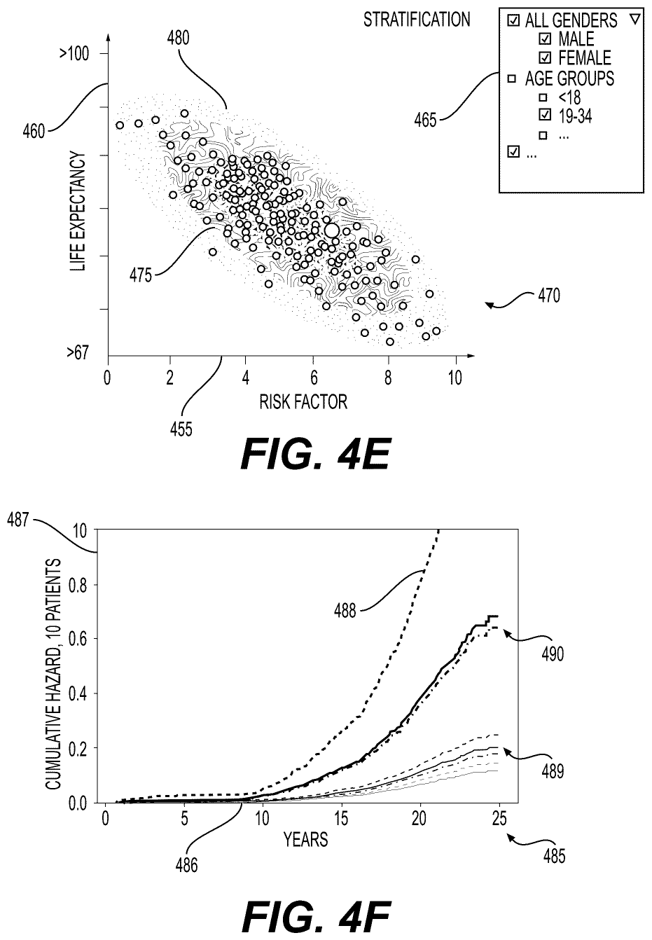

FIG. 4A illustrates a graph 400 of the time-dependent concordance 402 for the experimental model discussed above with regard to FIG. 2, whereby the x-axis 404 indicates the number of years after the last data sample was obtained in the longitudinal study (e.g., in a most recent patient visit of the study in which data was collected). The graph 400 shows the time-dependent accuracy of the model as measured by the c-index on the y-axis 406. The dotted line 408 indicates the overall concordance of the model. As shown above, predictions generally improve at longer time frames.

In one aspect, a single protein panel may be evaluated at a single time-point to make long-term predictions. In an example, a machine-learning model may be trained as an all-cause mortality model, and exhibited a high predictive accuracy in a timeframe 15-25 years using health data on adults aged 45-70. In a further aspect, one or more subsets of protein biomarkers or other health data may be used to train models for specific disease conditions or characteristics, such as to predict the likelihood that the person will develop Alzheimer's disease, heart disease, or other conditions.

A common confounder in healthcare prediction and evaluation is the presence of multiple health conditions, each of which can change plasma biomarkers and/or other characteristics. For example, a patient with autoimmunity may have inflammatory markers that could lead to an inaccurate prediction in other models that rely on inflammation. In an exemplary embodiment, patients may be stratified by health conditions to train not only comprehensive models, but also to train specific models using specific subsets of healthy patients or patients with pre-existing conditions. For example, different mortality/longevity models may be trained for patients having or not having diabetes, having or not having autoimmunity, etc. Different models for predicting Alzheimer's may be trained for patients with or without a history of a stroke. Moreover, although the previous examples were binary and involved patient stratification based on a single variable, it should be understood that patients may be stratified based on any number of variables into any number of segments. For instance, patients may be stratified based on age into 5 year segments, and then may be stratified further based on gender or any other characteristic(s). A bank of models trained with different strata of patients may thus be assembled.

In an example, concordance between a new patient and patients used to train different models may be evaluated to select a specific model that best matches to (e.g., is predictive of an outcome in a particular timeframe for) the new patient. For instance, if stratified models have considerably high predictive values, they may be used for inference rather than broader, less accurate models. Thus, in an exemplary methodology, patient data may be obtained, the patient data may be evaluated for concordance with a plurality of different models, each model being trained for a different stratification of patient and/or model characteristics, and a model with a highest concordance may be selected. The patient data may be evaluated using the selected model to generate a prediction for the patient.

In an example, multiple models may be deployed for inference with an algorithm to choose the model or models that most closely fit both patient characteristics and the desired prediction. For example, a 65-year old person with no underlying health conditions where short-term all-cause mortality is desired may have an inference run on a model trained on people aged 65+ that has a high concordance for short-term predictions. While the bank may contain some broadly trained models, it may also include models trained for composite predictions. In one embodiment, one or more short-term all-cause mortality model and a long-term all-cause mortality model may both be run, and for each time point in the prediction, the inference from the appropriate model with the highest time-dependent concordance may be selected.

One of the benefits of one or more aspects of this disclosure includes the use of a one-time measurement of biomarkers for a wide range of actionable predictions and time frames. For example, a single panel of biomarkers may be used to enrich patient medical data in order to simultaneously predict the presence or time frame of development of multiple health conditions.

As noted above, an exemplary output of a model trained and/or developed above may include a risk or hazard, which may be an over-all indicator or may be specific to a particular time window. Moreover, a prediction for a patient may be compared against similar predictions for other people and/or an overall average prediction for a population.

FIG. 4B depicts an exemplary illustration of a visual representation of output from a model trained according to one or more of the embodiments above. As shown in FIG. 4B, a patient-specific risk score 405 for a characteristic of interest, e.g., as predicted by a trained machine-learning model, may be plotted against risk assessments for a variety of patient strata, e.g., a first risk assessment 410 for a patient population as a whole and/or a second risk assessment 415 for a particular segment of the patient population such as a patient-specific segment (e.g., a grouping of patients having a threshold similarity to the patient of interest). As depicted in FIG. 4B, the risk assessments are illustrated as a gradient band that varies from a low risk to high. However, in various embodiments, any suitable type and kind of visual indication may be used.

FIG. 4C illustrates how the risk assessments for different patient population strata may vary relative to each other. For example, the risk assessments for patient population stratified by age tend to show a shift toward higher risk as age increases. Such risk assessments may enable more nuanced or granular consideration of a particular patient's risk, e.g., relative to an expected risk range notwithstanding their patient-specific medical information.

In another example, output from one or more model may be used to show predictions over a range of time steps. FIG. 4D illustrates how output from one or more all-cause mortality model may be used to generate a chart showing how a patient's predicted survival changes over time. In FIG. 4D, the x-axis 420 corresponds to time, and may be labeled based on time from a current date 425, a patient's age 430, or any other suitable axis label for time. The y-axis 430 corresponds to survival probability, e.g., the predicted probability that a person is still alive. The patient's predicted survival probability 435 may be plotted against other relevant metrics, such as an average survival probability 440 of patients having a threshold level of similarity with the patient (e.g., patients with similar demographics or the like). The region 445 corresponds to a first standard deviation from the patient's predicted survival probability 435 (e.g., a 67% prediction band), and the region 450 corresponds to a second standard deviation from the patient's predicted survival probability 435 (e.g., a 95% prediction band).

In another aspect, output from multiple patients may be used to generate demographic assessments and/or statistical analyses. In FIG. 4E, the x-axis 455 corresponds to a risk factor of a characteristic of interest, and the y-axis 460 corresponds to patient life expectancy. However, it should be understood that any suitable variable selection for axes may be made, e.g., for evaluating different characteristics and/or patient populations. An interface 465 illustrates how different strata of a patient population may be selected for inclusion in the plot 470. Regions 475 and 480 illustrate the first and second standard deviations, respectively, for the selected data in the plot 470. A highlighted data point 480 for the patient of interest may illustrate the patient's characteristics relative to the selected strata of the patient population.

Combinations of proteins analyzed according to one or more aspects of this disclosure may be more diagnostic than conventional analyses of single proteins. In one aspect, an interrelationship of proteins can shift with age, underlying health conditions, medications, and/or other factors

In a further aspect, a model such as one or more of the above may be used to make predictions about individual people, e.g., for multiple future points in time. Imputation may be performed by loading hyper-parameters of the model, and performing imputation on the required test values, demographic data, vitals, and medical history. The predictions provide a hazard at each time point in the series. The predictions can be expressed as a cumulative hazard function or as a % chance of survival at a given time. FIG. 4F illustrates a chart 485 that shows such predictions for multiple patients, in which the x-axis 486 corresponds to years form a current date, and the y-axis 487 corresponds to cumulative hazard, e.g., probability of patient death.

In FIG. 4F, cumulative hazard function of 10 patients in an experimental test data set is shown. A first patient 488 highly likely to die within 20 years of the data collection, evidenced by the line ending early at a cumulative hazard of 1.0 or 100%. The predictions ending around 0.2 or 20% represent patients 489 who will most likely be alive after 25 years. The lines ending at 0.6 or 60% represent patients 490 who are more likely to be deceased than alive after 25 years, though they are at lower risk of all-cause mortality than the patient 488.

A model, such as one or more of the foregoing, may be evaluated against several different test patients, with timeframe in terms of years after patient data was obtained. For example, the 10-year predictive accuracy of a model by comparing a prediction from the model based on 10-year old data with a patient's current condition. In one example, a 55-year old male smoker was predicted to have a 59% chance of survival at 21 years, and passed away 21.5 years after the patient data was obtained. A nonsmoking 58-year old female with lower biomarker levels was predicted to have a 90% chance of survival in the same timeframe, and she was still alive 25 years after her visit. In this manner, a model may be validated for different prediction time-frames.

One of the benefits of the techniques described in one or more of the embodiments above includes prediction and/or identification of conditions such as chronic disease that might have been missed by conventional assessments, or prediction and/or identification of such conditions earlier than may have been possible by conventional assessment, such as when a condition is pre-symptomatic. In an example, by enriching conventional patient medical health information with protein biomarker data, machine-learning models trained via techniques such as those discussed above may be configured to perform proteomic analysis that accounts for a far more interconnected and intricate understanding of the relations of proteins and health characteristics than is possible in conventional solutions. In one aspect, protein presence in the body may not only be causal, like genes which may affect development of characteristics, but also reactive to other health characteristics, and so may be a for more dynamic indication of patient health than other biomarkers.

In an experimental assessment, patient medical data was collected from various sources, and protein biomarker data was used to enrich conventional clinical data. A machine-learning model was trained using the collected data, and was assessed using a validation population having a 10% prevalence rate of a characteristic of interest. The model was found to have a ˜90% sensitivity to the characteristic of interest, and a ˜90-95% specificity, and exhibited an ROC-AUC score of 0.9 with a ˜67% positive prediction rate and a ˜98% negative prediction rate. These results illustrate how the techniques discussed above may be used to rapidly and efficiently spin up models with at least 90% performance for any particular characteristic of interest.

In one aspect, the techniques discussed above enable consideration of biomarkers not conventionally considered during clinical diagnoses. In an example, biomarkers for renal function, neuro-hormonal function, inflammatory response, Myocyte stress, Myocyte injury, fibrosis, to name a few, enable a combination of novel and conventional biomarkers to improve prognostic value of predictions for patients. Biomarkers may also be sourced from previously unconventional sources, such as wearable technologies, prescription data, etc., that may not be fully considered in a conventional solution.

Further aspects of one or machine-learning model including one or more aspects according to this disclosure, and/or how such a model may be generated or utilized to generate predictions of one or more patient health characteristics are discussed in further detail in the methods below.

In the methods disclosed herein, various acts may be described as performed or executed by a component from FIG. 1. However, it should be understood that in various embodiments, various components of the environment 100 discussed above may execute instructions or perform acts including the acts discussed below. An act performed by a device may be considered to be performed by a processor, actuator, or the like associated with that device. Further, it should be understood that in various embodiments, various steps may be added, omitted, or rearranged in any suitable manner.

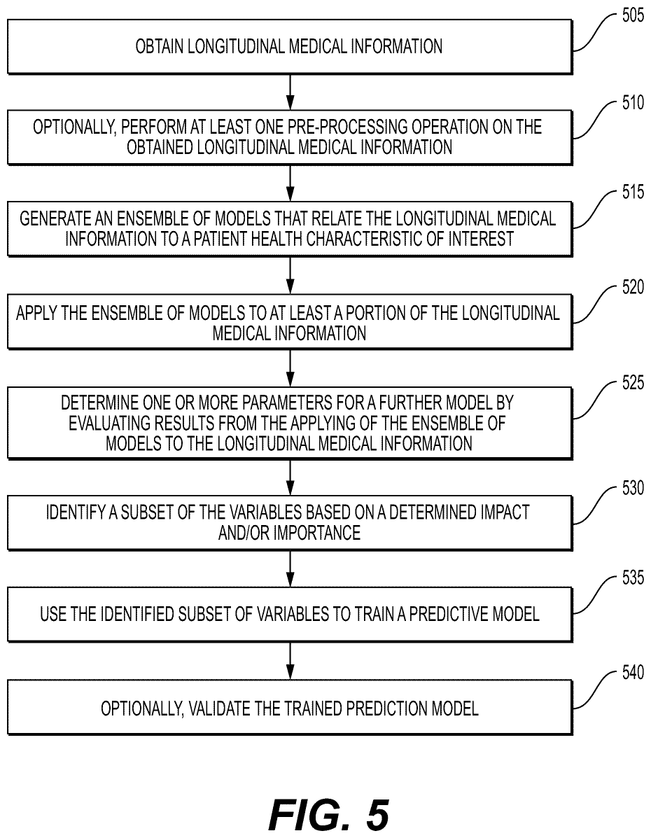

FIG. 5 illustrates an exemplary process for generating and/or training one or more machine-learning model to generate a prediction of a patient health characteristic of interest, such as in the various examples discussed above. At step 505, longitudinal medical information may be obtained, e.g., via a model system 115 or the like. Such information may be obtained from one or more sources, e.g., a data store 110, a provider system 125, or the like. The longitudinal medical information may include medical information for a plurality of patients. The information may include, for example, demographic information, patient characteristics, medical history or health status, outcome data, lab result data, lifestyle history, heart health, risk factors, biomarkers (e.g. protein biomarkers), timing and severity of cardiovascular diseases, and/or eventually mortality and primary cause of death. In an example, the information may include, for each patient of the plurality of patients, and for each time-step over a longitudinal study, a respective value for variables such as the foregoing. While having the same variables for each patient and/or for each longitudinal time step may be beneficial, such consistency may not be available. For instance, earlier testing in a longitudinal study may not have captured a variable that was added later, or a particular patient may have missed an appointment, or the like.

Optionally, at step 510, at least one pre-processing operation may be performed on the obtained information. In one example, the pre-processing operation may include a down-sampling operation. For instance, a sub-set of the plurality of patients may be resampled to equalize a sample size with a relatively small quantity of patients exhibiting a characteristic of interest. In another example, the pre-processing operation may include a variable pruning operation. For instance, each variable included in the obtained information may be evaluated for missing entries. A variable missing more than a threshold number of entries may be pruned from the information.

In another instance, the model system 115 or the like may determine that a subset of the variables are one or more of correlated, deterministic of each other, or descriptive of each other. In other words, the values of some variables may be informative of or informed by others. For instance, a first variable may include “smoking: y/n” and a second variable may include “number of cigarettes/day.” Such variables are related. However, one is more information-dense than the other. Specifically, the second variable is quantitative, while the first variable is qualitative, and thus in some embodiments the less information-dense of a set of related variables may be pruned. Generally, quantitative data is more information-dense than qualitative. In another instance, multiple variables may be quantitative, but have different levels of granularity, e.g., drinks per day vs. drinks per month. A more granular quantity, e.g., drinks per day, is generally more information dense than a less granular quantity. In some instances, a plurality of variables may inform another variable or variables. For instance, data regarding exercise time/day, distance walked/day, etc., may be related to an overall fitness time variable. In some instances, the plurality of variables may be pruned in favor of such a variable, e.g., to reduce a total quantity of variables. In some embodiments, the loss of information, e.g., by selecting less information dense variables, may be balanced against the reduction in complexity of the data by removal of variables. In another example, variables may be compared as to which has fewer missing entries/values. In other words, instead of or in addition to determining which variable is more information dense, or determining which variable adds to complexity, the model system 115 may determine which variable is better represented in the obtained data set.

In a further example, the pre-processing operation may include an interpolation operation. In on example, an interpolation operation includes determining a value or values for a variable based on another value or values of that variable. For instance, if a patient answered the question of “cigarettes/day” with “4” on a first patient visit in year X, there is no recorded answer for a second visit in year X+1, and the patient answered with “6” on a third visit in year X+2, the model system 115 may interpolate the answers surrounding the missing entry and generate an interpolated answer of “5.” In another example, another variable or variables may be used to interpolate a value or values for a variable. For example, values for blood pressure, physical fitness, exercise, cholesterol, etc., may be used to generate a value for a heart health variable, etc. In various embodiments, interpolation may be performed before or after a pruning operation. Performing interpolation after a pruning operation may reduce an amount of interpolation performed, and/or facilitate preservation of the integrity of the obtained data.

It should be understood that such pre-processing operations are illustrative, and that any suitable operation or combination of operations may be used.

At step 515, the model system 115 may generate an ensemble of models that relate the longitudinal medical information to a patient health characteristic of interest. In various embodiments, one or more model parameters may be varied across the ensemble such as, for example, model type, learning rate, sampling type, number of estimators, hidden layers, trees, etc., parameters used, or the like. In an example, variation of the model type may include using one or more of a linear model, a least-square or partial least-square regression model, a recursive partitioning model such as a Cox model, a gradient boosted Cox model, a Cox Random Forest Model, etc., a neural network, e.g., a deep neural network, a support vector machine, a Naïve Bayes model, etc.

At step 520, the ensemble of models may be applied to at least a portion of the longitudinal medial information. For example, the ensemble may be applied to a portion or portions of the information sequestered as training data, pre-training data, or the like.

At step 525, the model system 115 may determine one or more parameters for a further model by evaluating results from the applying of the ensemble of models to the longitudinal medical information. In one example, the ensemble of models may be evaluated, e.g., by comparison with labels or ground truth applied to the medical information, to determine which models of the ensemble were more accurate, which may then inform an optimal setting for the parameter or parameters varied across the ensemble. For instance, in an ensemble in which the variables used was varied, the results may inform which variables in the medical information tend to lead to more accurate results. In another example, an ensemble in which the variables were constant, but in which model type was varied may inform as which type of model may be more applicable to representing the medical information.

In a further example, the evaluating of the results of the ensemble may be used to determine one or more of an impact or importance of the variables of the longitudinal medical information. In an example, evaluating results from applying the ensemble of models to the longitudinal medical information includes a univariate analysis of the variables to determine the impact. Such a univariate analysis may be used to determine which of the variables from the medical information may be correlated with the characteristic of interest by itself. Variables that are correlated with the characteristic of interest, either positively or negatively, may provide predictive value. However, variables which are not very correlated would provide little impact on a predictive model. Thus, in some embodiments, variables having a relatively low impact, based on the univariate analysis, may be pruned from the data set.

In some embodiments, evaluating results from applying the ensemble of models to the longitudinal medical information includes a multivariate analysis of the variables to determine the importance. In an example, a plurality of different model types may be applied to a set of training data sampled from the medical information. Results may be evaluated to compare the accuracy of the different model types, and/or to evaluate the importance of the variables in the medical information relative to each other.

At step 530, the model system 115 may identify a subset of the variables based on the determined one or more of the impact or importance. In an example, the importance of the variables may be compared against the impact determined via univariate analysis above. In an example, a higher importance value and a higher impact may be indicative that a particular variable is relevant across a variety of models. Such relevant variables may thus be good candidates for use in the predictive model. In some embodiments, a plurality of relevant variables are identified.

At step 535, the subset of the variables may be used, e.g., via the model system 115, to train a predictive model. In an example, a type and/or other parameters of the predictive model is based on the results of one or more of the ensemble analysis or the multivariate analysis.

Optionally, at step 540, the trained prediction model may be validated, e.g., by applying the prediction model to a portion of the medical information sequestered as validation data and comparing the results to corresponding labels or ground truth.

In various embodiments, the variables used in the method above, e.g., the variables obtained at step 505, evaluated at step 525, identified at step 530, and/or used to train the prediction model at step 535 may further be selected based on the patient medical characteristic of interest. In an example, different protein biomarkers may be more or less predictive of various patient medical characteristics. In various embodiments, variables for protein biomarkers any one or more of the foregoing steps may be selected from the listings of protein biomarkers, groupings of protein biomarkers, or categories of protein biomarkers included in Tables A-R.

In an example, one or more protein biomarker may be identified from each of a plurality of the following categories: Cell Cycle and Division; Cell Death and Autophagy; Cell Movement and Transport; Cell Structure and Adhesion; Chromatin and Gene Regulation; Cilia and Microtubules; Development and Differentiation; DNA and RNA Processes; Immune and Defense; Metabolic Processes; Organ and System Processes; Protein Processes; Signaling and Communication; and Wound Healing and Repair.

In an example, for a predictive model configured to generate predictions for longevity or all-cause mortality, one or more protein biomarker may be identified from each of a plurality of the following groupings identified via their biological process as indexed by UniPoint Gene Ontology (GO): mitotic cell cycle (GO:0000278); mitotic nuclear division (GO:0140014); chromosome segregation (GO:0007059); programmed cell death (GO:0012501); autophagy (GO:0006914); cell motility GO:0048870); vesicle-mediated transport (GO:0016192); transmembrane transport GO:0055085); intracellular protein transport (GO:0006886); nucleocytoplasmic transport (GO:0006913); cell adhesion (GO:0007155); extracellular matrix organization (GO:0030198); cell junction organization (GO:0034330); cytoskeleton organization (GO:0007010); membrane organization (GO:0061024); mitochondrion organization (GO:0007005); chromatin organization (GO:0006325); microtubule-based movement (GO:0007018); cilium organization (GO:0044782); anatomical structure development (GO:0048856); cell differentiation (GO:0030154); reproductive process (GO:0022414); nervous system process (GO:0050877); circulatory system process (GO:0003013); regulation of DNA-templated transcription (GO:0006355); mRNA metabolic process (GO:0016071); DNA recombination (GO:0006310); DNA-templated transcription (GO:0006351); regulatory ncRNA-mediated gene silencing GO:0031047); mitochondrial gene expression (GO:0140053); immune system process (GO:0002376); inflammatory response (GO:0006954); defense response to other organism (GO:0098542); lipid metabolic process (GO:0006629); carbohydrate derivative metabolic process (GO:1901135); protein catabolic process (GO:0030163); carbohydrate metabolic process (GO:0005975); nucleobase-containing small molecule metabolic process (GO:0055086); generation of precursor metabolites and energy (GO:0006091); cellular modified amino acid metabolic process (GO:0006575); sulfur compound metabolic process (GO:0006790); detoxification (GO:0098754); vitamin metabolic process (GO:0006766); amino acid metabolic process (GO:0006520); muscle system process (GO:0003012); renal system process (GO:0003014); endocrine process (GO:0050886); digestive system process (GO:0022600); protein-containing complex assembly (GO:0065003); protein maturation (GO:0051604); protein folding (GO:0006457); protein glycosylation (GO:0006486); signaling (GO:0023052); protein localization to plasma membrane (GO:0072659); and wound healing (GO:0042060).

In an example, for a predictive model configured to generate predictions for heart function, one or more protein biomarker may be identified from each of a plurality of the following groupings: mitotic cell cycle (GO:0000278); mitotic nuclear division (GO:0140014); meiotic nuclear division (GO:0140013); chromosome segregation (GO:0007059); cytokinesis (GO:0000910); programmed cell death GO:0012501); autophagy (GO:0006914); cell motility (GO:0048870); vesicle-mediated transport (GO:0016192); transmembrane transport (GO:0055085); intracellular protein transport (GO:0006886); nucleocytoplasmic transport (GO:0006913); cell adhesion (GO:0007155); cytoskeleton organization (GO:0007010); cell junction organization (GO:0034330); extracellular matrix organization (GO:0030198); membrane organization (GO:0061024); mitochondrion organization GO:0007005); establishment or maintenance of cell polarity (GO:0007163); telomere organization (GO:0032200); chromatin organization (GO:0006325); microtubule-based movement (GO:0007018); cilium organization (GO:0044782); anatomical structure development (GO:0048856); cell differentiation (GO:0030154); reproductive process (GO:0022414); circulatory system process (GO:0003013); nervous system process (GO:0050877); regulation of DNA-templated transcription (GO:0006355); DNA-templated transcription (GO:0006351); DNA recombination (GO:0006310); mRNA metabolic process (GO:0016071); DNA repair (GO:0006281); regulatory ncRNA-mediated gene silencing (GO:0031047); DNA replication (GO:0006260); tRNA metabolic process (GO:0006399); mitochondrial gene expression (GO:0140053); immune system process (GO:0002376); inflammatory response (GO:0006954); defense response to other organism (GO:0098542); lipid metabolic process (GO:0006629); protein catabolic process (GO:0030163); carbohydrate derivative metabolic process (GO:1901135); carbohydrate metabolic process (GO:0005975); nucleobase-containing small molecule metabolic process (GO:0055086); generation of precursor metabolites and energy (GO:0006091); detoxification (GO:0098754); vitamin metabolic process (GO:0006766); cellular modified amino acid metabolic process (GO:0006575); amino acid metabolic process (GO:0006520); sulfur compound metabolic process (GO:0006790); muscle system process (GO:0003012); digestive system process (GO:0022600); endocrine process (GO:0050886); renal system process (GO:0003014); protein-containing complex assembly (GO:0065003); protein maturation (GO:0051604); protein folding (GO:0006457); protein glycosylation (GO:0006486); signaling (GO:0023052); protein localization to plasma membrane (GO:0072659); and wound healing (GO:0042060).

In an example, for a predictive model configured to generate predictions for chronic kidney disease, one or more protein biomarker may be identified from each of a plurality of the following groupings: mitotic cell cycle (GO:0000278); mitotic nuclear division (GO:0140014); meiotic nuclear division (GO:0140013); chromosome segregation (GO:0007059); cytokinesis (GO:0000910); programmed cell death GO:0012501); autophagy (GO:0006914); cell motility (GO:0048870); vesicle-mediated transport (GO:0016192); transmembrane transport (GO:0055085); intracellular protein transport (GO:0006886); nucleocytoplasmic transport (GO:0006913); cell adhesion (GO:0007155); cytoskeleton organization (GO:0007010); cell junction organization (GO:0034330); extracellular matrix organization (GO:0030198); membrane organization (GO:0061024); mitochondrion organization GO:0007005); establishment or maintenance of cell polarity (GO:0007163); telomere organization (GO:0032200); chromatin organization (GO:0006325); microtubule-based movement (GO:0007018); cilium organization (GO:0044782); anatomical structure development (GO:0048856); cell differentiation (GO:0030154); reproductive process (GO:0022414); circulatory system process (GO:0003013); nervous system process (GO:0050877); regulation of DNA-templated transcription (GO:0006355); DNA-templated transcription (GO:0006351); DNA recombination (GO:0006310); mRNA metabolic process (GO:0016071); DNA repair (GO:0006281); regulatory ncRNA-mediated gene silencing (GO:0031047); DNA replication (GO:0006260); tRNA metabolic process (GO:0006399); mitochondrial gene expression (GO:0140053); immune system process (GO:0002376); inflammatory response (GO:0006954); defense response to other organism (GO:0098542); lipid metabolic process (GO:0006629); protein catabolic process (GO:0030163); carbohydrate derivative metabolic process (GO:1901135); carbohydrate metabolic process (GO:0005975); nucleobase-containing small molecule metabolic process (GO:0055086); generation of precursor metabolites and energy (GO:0006091); detoxification (GO:0098754); vitamin metabolic process (GO:0006766); cellular modified amino acid metabolic process (GO:0006575); amino acid metabolic process (GO:0006520); sulfur compound metabolic process (GO:0006790); muscle system process (GO:0003012); digestive system process (GO:0022600); endocrine process (GO:0050886); renal system process (GO:0003014); protein-containing complex assembly (GO:0065003); protein maturation (GO:0051604); protein folding (GO:0006457); protein glycosylation (GO:0006486); signaling (GO:0023052); protein localization to plasma membrane (GO:0072659); and wound healing (GO:0042060).

In an example, for a predictive model configured to generate predictions for chronic obstructive pulmonary disease, one or more protein biomarker may be identified from each of a plurality of the following groupings: mitotic cell cycle (GO:0000278); mitotic nuclear division (GO:0140014); meiotic nuclear division GO:0140013); chromosome segregation (GO:0007059); cytokinesis (GO:0000910); programmed cell death (GO:0012501); autophagy (GO:0006914); cell motility (GO:0048870); vesicle-mediated transport (GO:0016192); transmembrane transport GO:0055085); intracellular protein transport (GO:0006886); nucleocytoplasmic transport (GO:0006913); cell adhesion (GO:0007155); cytoskeleton organization GO:0007010); cell junction organization (GO:0034330); extracellular matrix organization (GO:0030198); membrane organization (GO:0061024); mitochondrion organization (GO:0007005); establishment or maintenance of cell polarity (GO:0007163); telomere organization (GO:0032200); chromatin organization GO:0006325); microtubule-based movement (GO:0007018); cilium organization GO:0044782); anatomical structure development (GO:0048856); cell differentiation GO:0030154); reproductive process (GO:0022414); circulatory system process (GO:0003013); nervous system process (GO:0050877); regulation of DNA-templated transcription (GO:0006355); DNA-templated transcription (GO:0006351); DNA recombination (GO:0006310); mRNA metabolic process (GO:0016071); DNA repair GO:0006281); regulatory ncRNA-mediated gene silencing (GO:0031047); DNA replication (GO:0006260); tRNA metabolic process (GO:0006399); mitochondrial gene expression (GO:0140053); immune system process (GO:0002376); inflammatory response (GO:0006954); defense response to other organism GO:0098542); lipid metabolic process (GO:0006629); protein catabolic process GO:0030163); carbohydrate derivative metabolic process (GO:1901135); carbohydrate metabolic process (GO:0005975); nucleobase-containing small molecule metabolic process (GO:0055086); generation of precursor metabolites and energy (GO:0006091); detoxification (GO:0098754); vitamin metabolic process (GO:0006766); cellular modified amino acid metabolic process (GO:0006575); amino acid metabolic process (GO:0006520); sulfur compound metabolic process (GO:0006790); muscle system process (GO:0003012); digestive system process (GO:0022600); endocrine process (GO:0050886); renal system process (GO:0003014); protein-containing complex assembly (GO:0065003); protein maturation (GO:0051604); protein folding GO:0006457); protein glycosylation (GO:0006486); signaling (GO:0023052); protein localization to plasma membrane (GO:0072659); and wound healing (GO:0042060).

In an example, for a predictive model configured to generate predictions for dementia, one or more protein biomarker may be identified from each of a plurality of the following groupings: mitotic cell cycle (GO:0000278); mitotic nuclear division (GO:0140014); meiotic nuclear division (GO:0140013); chromosome segregation GO:0007059); cytokinesis (GO:0000910); programmed cell death (GO:0012501); autophagy (GO:0006914); cell motility (GO:0048870); vesicle-mediated transport GO:0016192); transmembrane transport (GO:0055085); intracellular protein transport (GO:0006886); nucleocytoplasmic transport (GO:0006913); cell adhesion GO:0007155); cytoskeleton organization (GO:0007010); cell junction organization GO:0034330); extracellular matrix organization (GO:0030198); mitochondrion organization (GO:0007005); membrane organization (GO:0061024); establishment or maintenance of cell polarity (GO:0007163); telomere organization (GO:0032200); chromatin organization (GO:0006325); microtubule-based movement (GO:0007018); cilium organization (GO:0044782); anatomical structure development (GO:0048856); cell differentiation (GO:0030154); reproductive process (GO:0022414); circulatory system process (GO:0003013); nervous system process (GO:0050877); muscle system process (GO:0003012); regulation of DNA-templated transcription (GO:0006355); DNA-templated transcription (GO:0006351); DNA recombination GO:0006310); mRNA metabolic process (GO:0016071); DNA repair (GO:0006281); regulatory ncRNA-mediated gene silencing (GO:0031047); DNA replication GO:0006260); tRNA metabolic process (GO:0006399); mitochondrial gene expression (GO:0140053); immune system process (GO:0002376); inflammatory response (GO:0006954); defense response to other organism (GO:0098542); lipid metabolic process (GO:0006629); protein catabolic process (GO:0030163); carbohydrate derivative metabolic process (GO:1901135); carbohydrate metabolic process (GO:0005975); nucleobase-containing small molecule metabolic process GO:0055086); generation of precursor metabolites and energy (GO:0006091); detoxification (GO:0098754); vitamin metabolic process (GO:0006766); cellular modified amino acid metabolic process (GO:0006575); amino acid metabolic process GO:0006520); sulfur compound metabolic process (GO:0006790); digestive system process (GO:0022600); endocrine process (GO:0050886); renal system process GO:0003014); protein-containing complex assembly (GO:0065003); protein maturation (GO:0051604); protein folding (GO:0006457); protein glycosylation GO:0006486); signaling (GO:0023052); protein localization to plasma membrane GO:0072659); and wound healing (GO:0042060).

In an example, for a predictive model configured to generate predictions for Alzheimer's disease-related plaque formation, one or more protein biomarker may be identified from each of a plurality of the following groupings: mitotic cell cycle GO:0000278); mitotic nuclear division (GO:0140014); meiotic nuclear division GO:0140013); chromosome segregation (GO:0007059); cytokinesis (GO:0000910); programmed cell death (GO:0012501); autophagy (GO:0006914); cell motility (GO:0048870); vesicle-mediated transport (GO:0016192); transmembrane transport GO:0055085); intracellular protein transport (GO:0006886); nucleocytoplasmic transport (GO:0006913); cell adhesion (GO:0007155); cytoskeleton organization GO:0007010); cell junction organization (GO:0034330); extracellular matrix organization (GO:0030198); mitochondrion organization (GO:0007005); membrane organization (GO:0061024); establishment or maintenance of cell polarity (GO:0007163); telomere organization (GO:0032200); chromatin organization GO:0006325); microtubule-based movement (GO:0007018); cilium organization GO:0044782); anatomical structure development (GO:0048856); cell differentiation GO:0030154); reproductive process (GO:0022414); nervous system process (GO:0050877); circulatory system process (GO:0003013); muscle system process GO:0003012); regulation of DNA-templated transcription (GO:0006355); DNA-templated transcription (GO:0006351); DNA recombination (GO:0006310); mRNA metabolic process (GO:0016071); DNA repair (GO:0006281); regulatory ncRNA-mediated gene silencing (GO:0031047); DNA replication (GO:0006260); tRNA metabolic process (GO:0006399); mitochondrial gene expression (GO:0140053); immune system process (GO:0002376); inflammatory response (GO:0006954); defense response to other organism (GO:0098542); lipid metabolic process (GO:0006629); protein catabolic process (GO:0030163); carbohydrate derivative metabolic process (GO:1901135); carbohydrate metabolic process (GO:0005975); nucleobase-containing small molecule metabolic process (GO:0055086); generation of precursor metabolites and energy (GO:0006091); detoxification (GO:0098754); vitamin metabolic process (GO:0006766); cellular modified amino acid metabolic process (GO:0006575); amino acid metabolic process (GO:0006520); sulfur compound metabolic process (GO:0006790); digestive system process (GO:0022600); endocrine process (GO:0050886); renal system process (GO:0003014); protein-containing complex assembly (GO:0065003); protein maturation (GO:0051604); protein folding (GO:0006457); protein glycosylation (GO:0006486); signaling (GO:0023052); protein localization to plasma membrane (GO:0072659); and wound healing (GO:0042060).

Further examples, such as examples of proteins included in the categories, and GO groupings listed above are included in Tables A-R. Moreover, while the number of individual proteins, GO groups, and even categories makes for a large potential space for variable selection, one of ordinary skill in the art would recognize that the processes for selection from this potential space discussed in this disclosure, e.g., via the ensemble model operations, univariate and multivariate analyses, etc., are important technical advances, especially in view of continued discovery of new proteins, new biological relevancy of proteins, GO groupings, and protein categories, etc. In an example, while examples are provided herein for biomarkers relevant to an illustrative selection of diseases or disorders, applying one or more of the techniques disclosed herein enables the arrival of a similar dataset for any disease or characteristic of interest. In this manner, a predictive model may be generated for any characteristic or characteristics of interest.

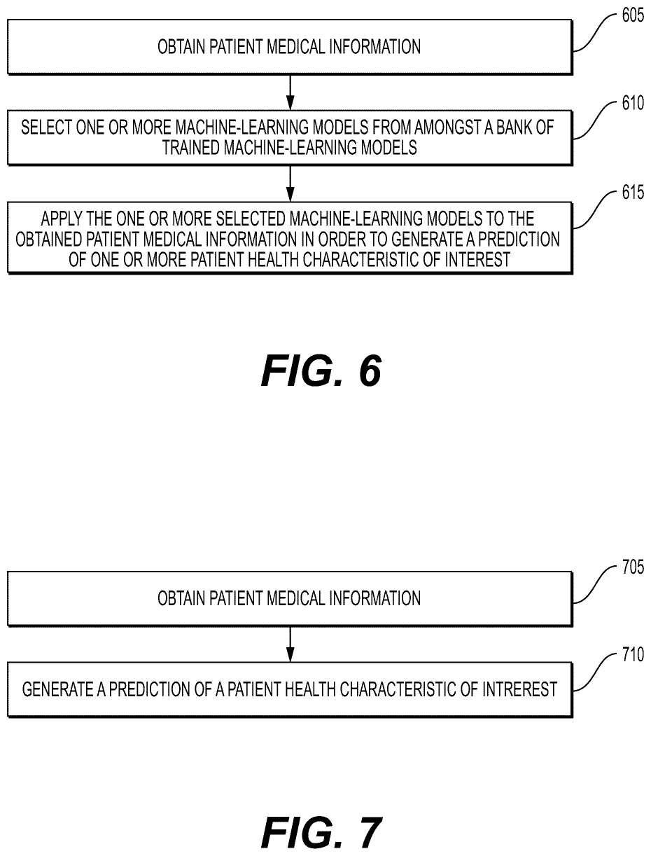

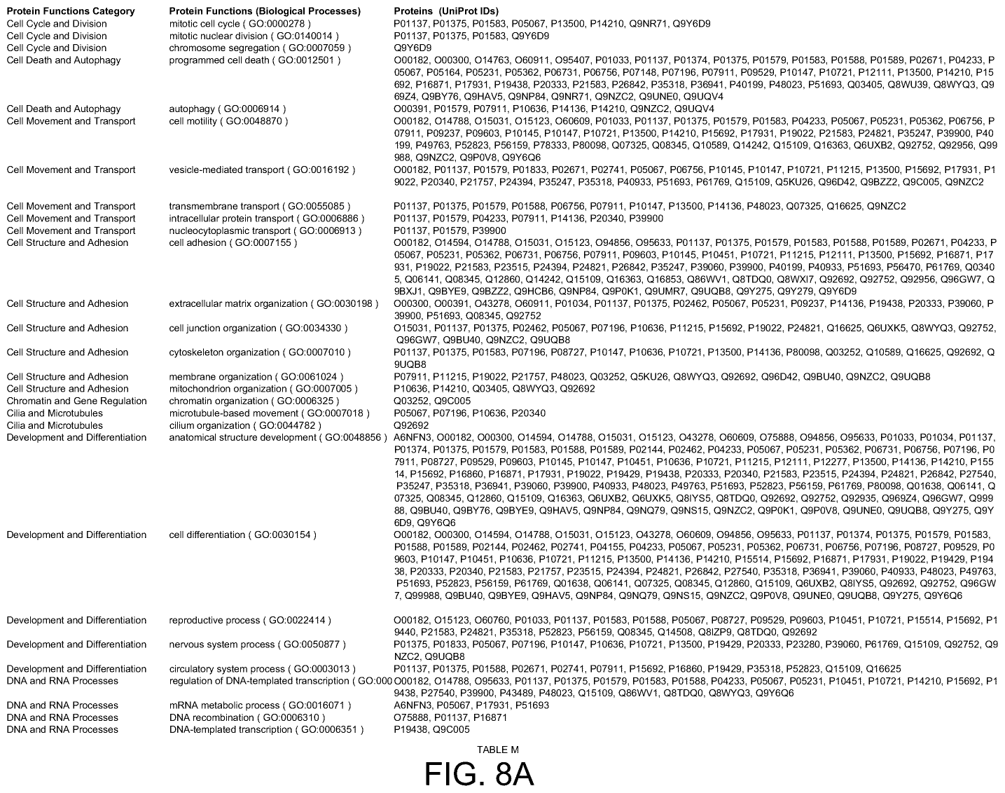

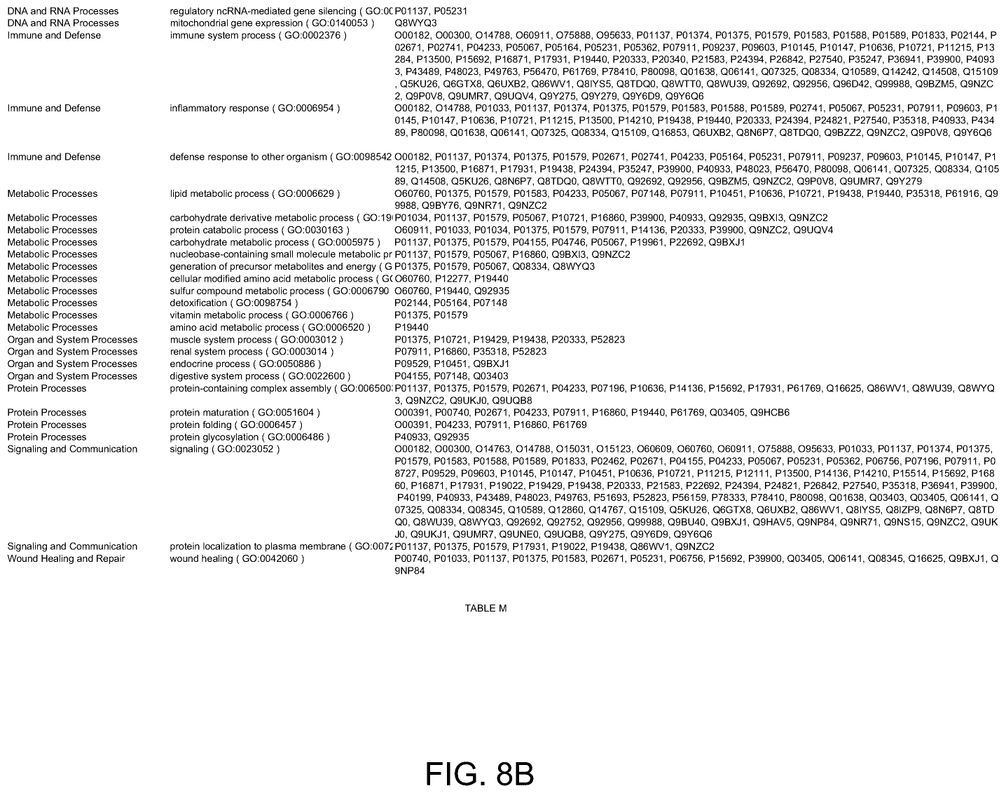

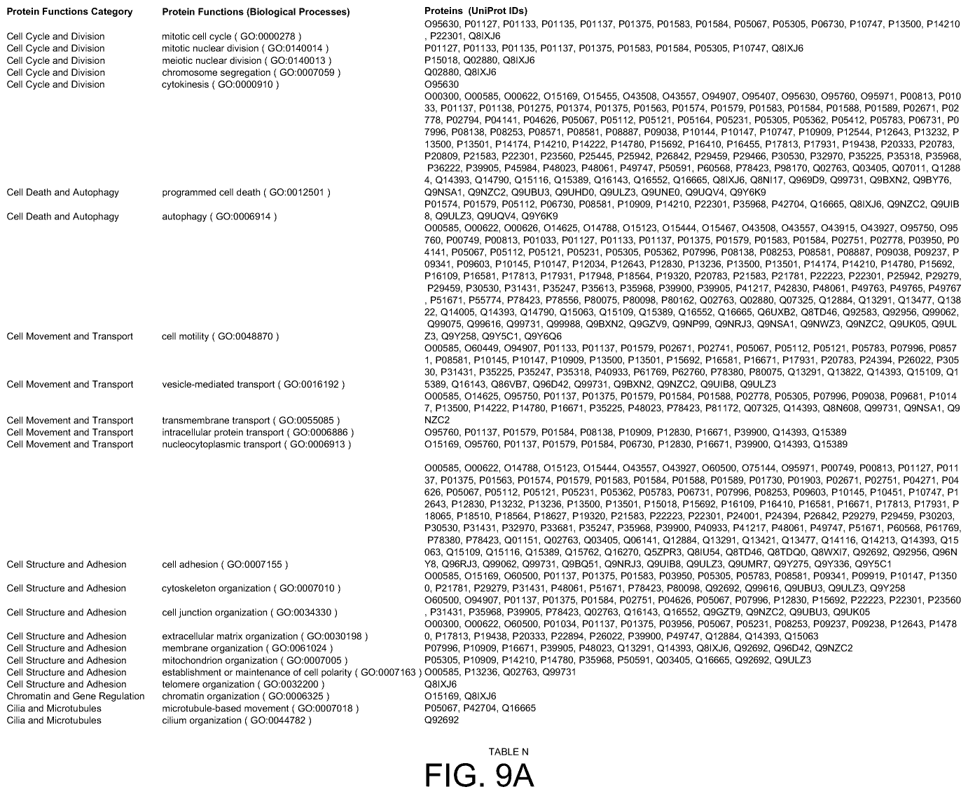

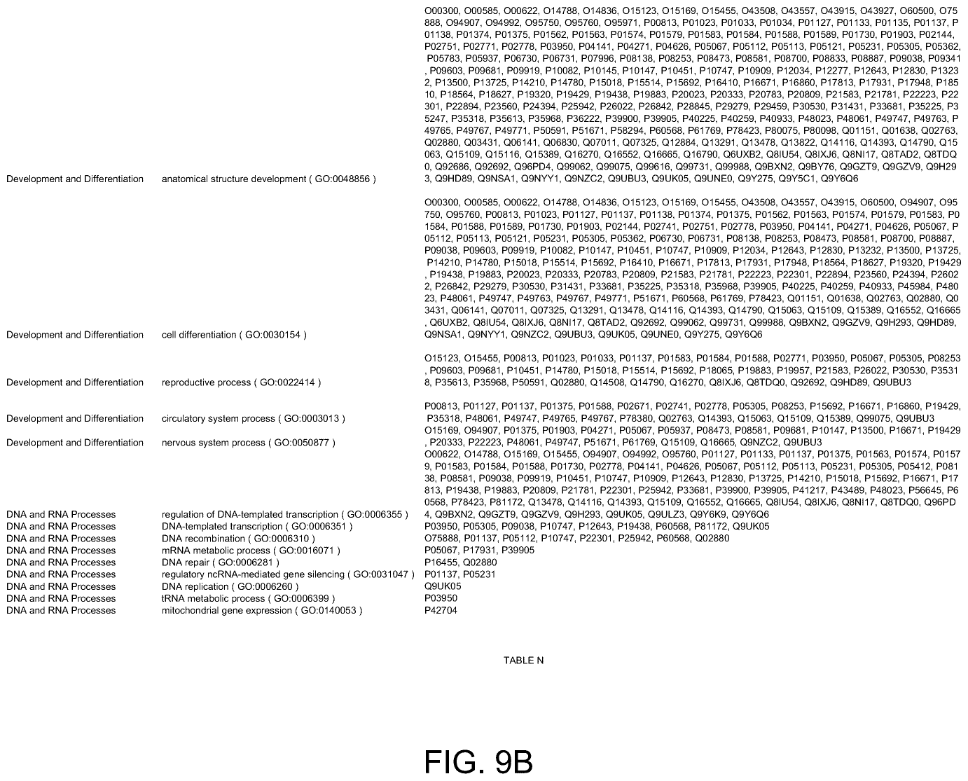

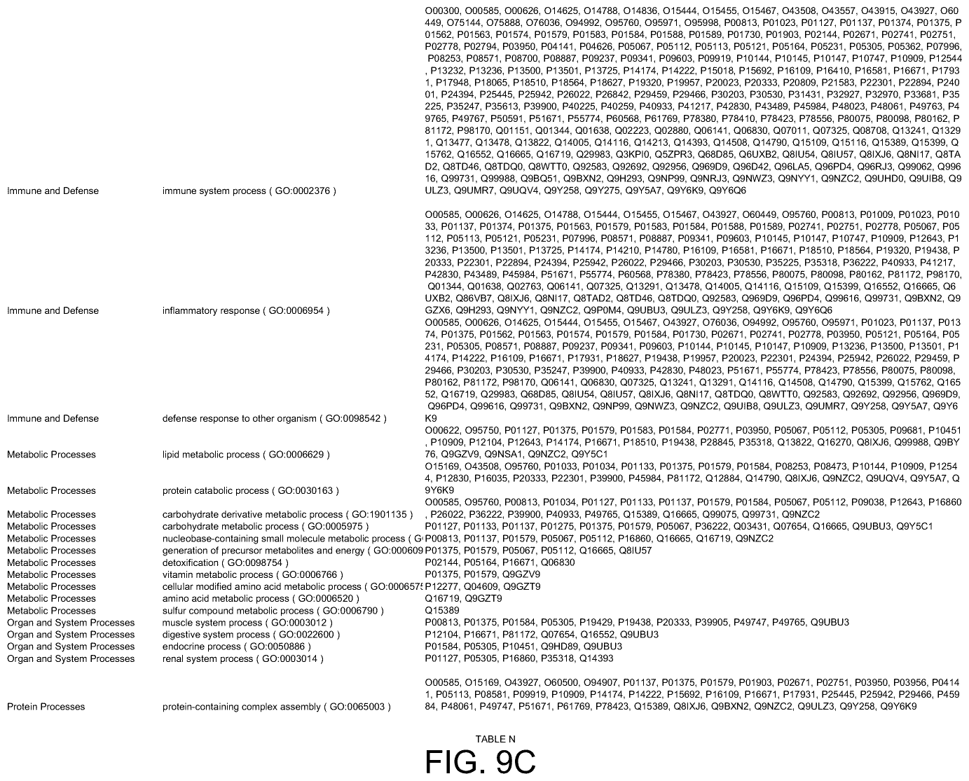

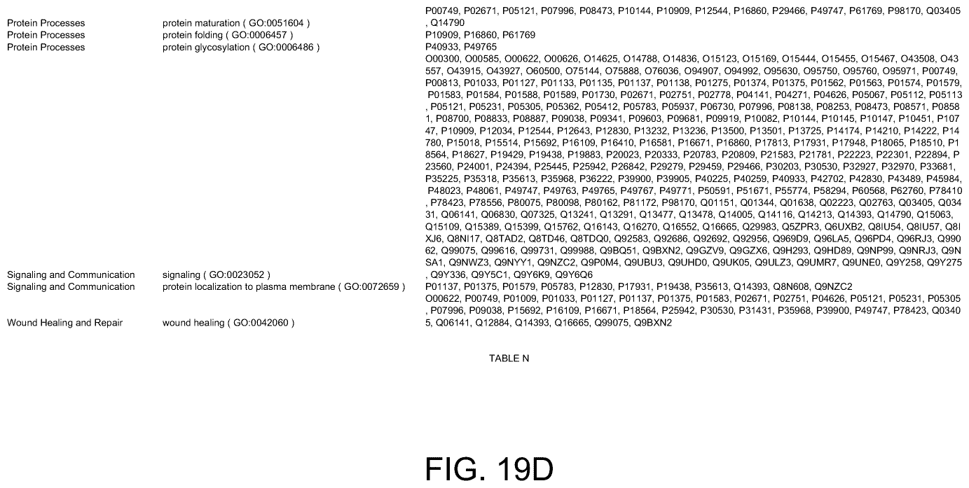

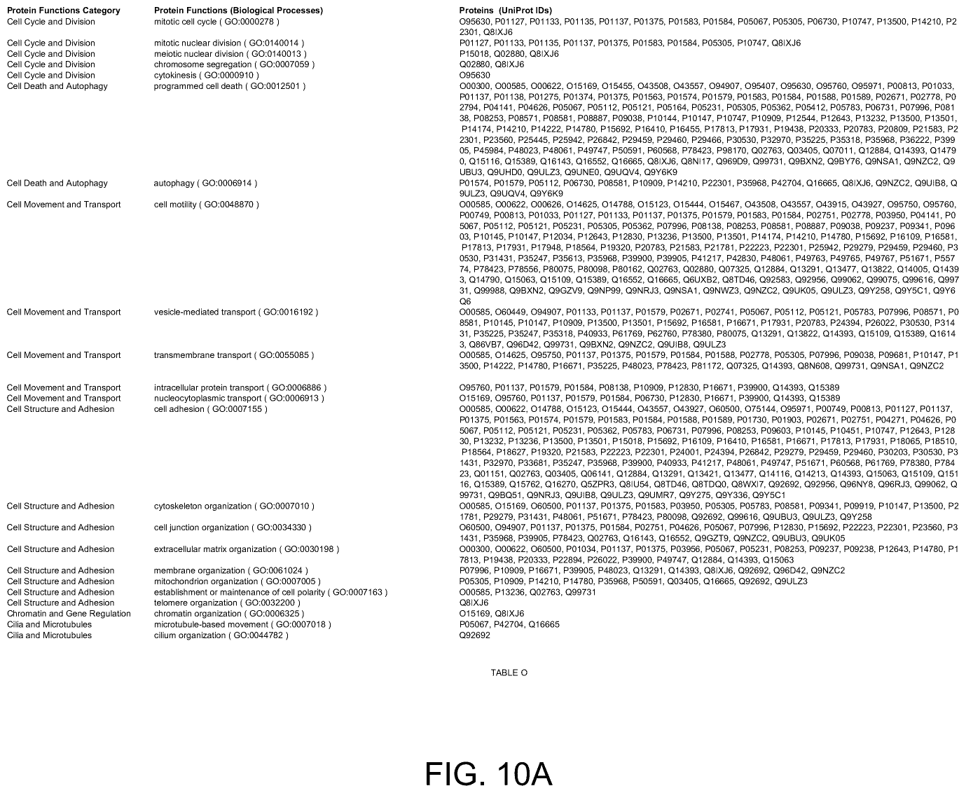

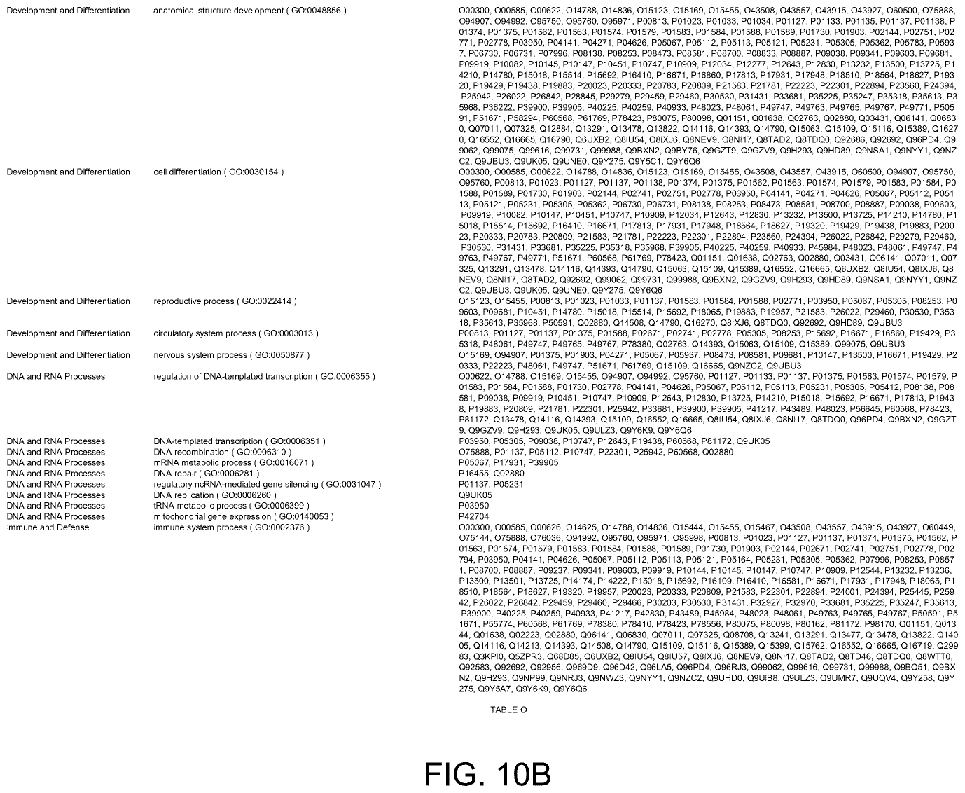

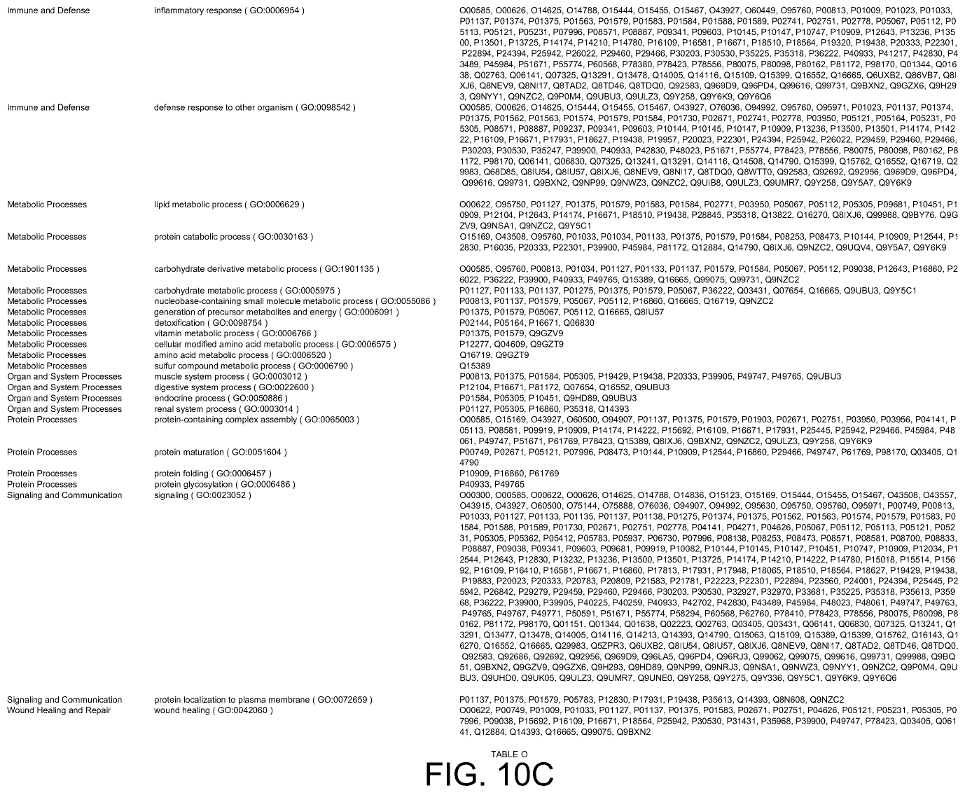

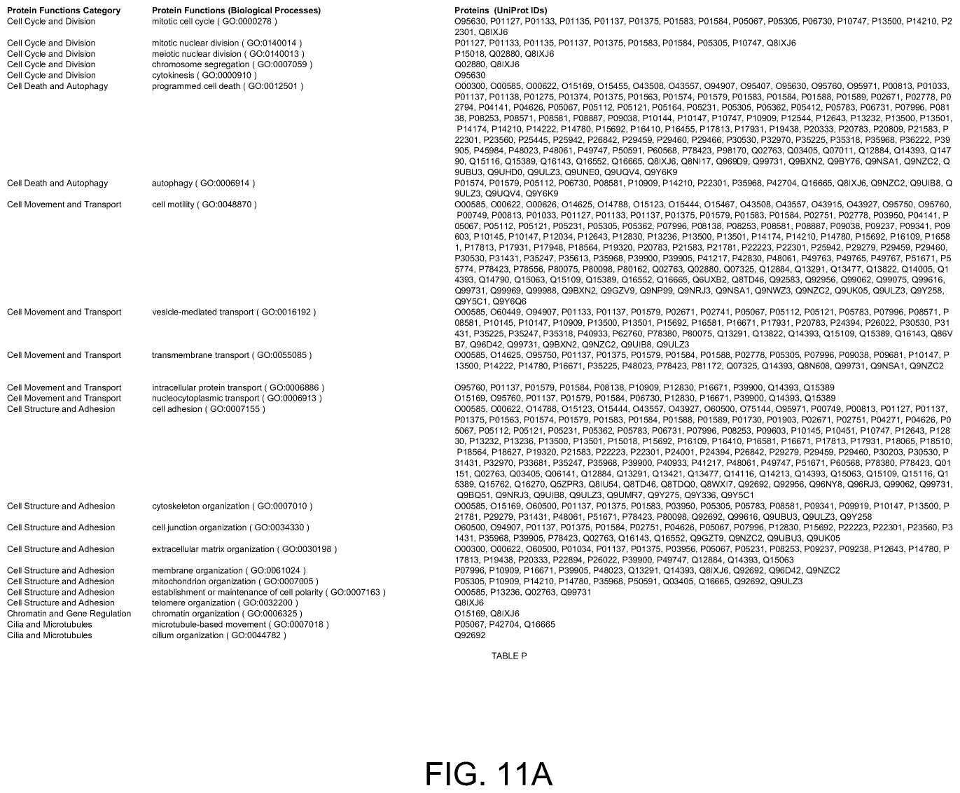

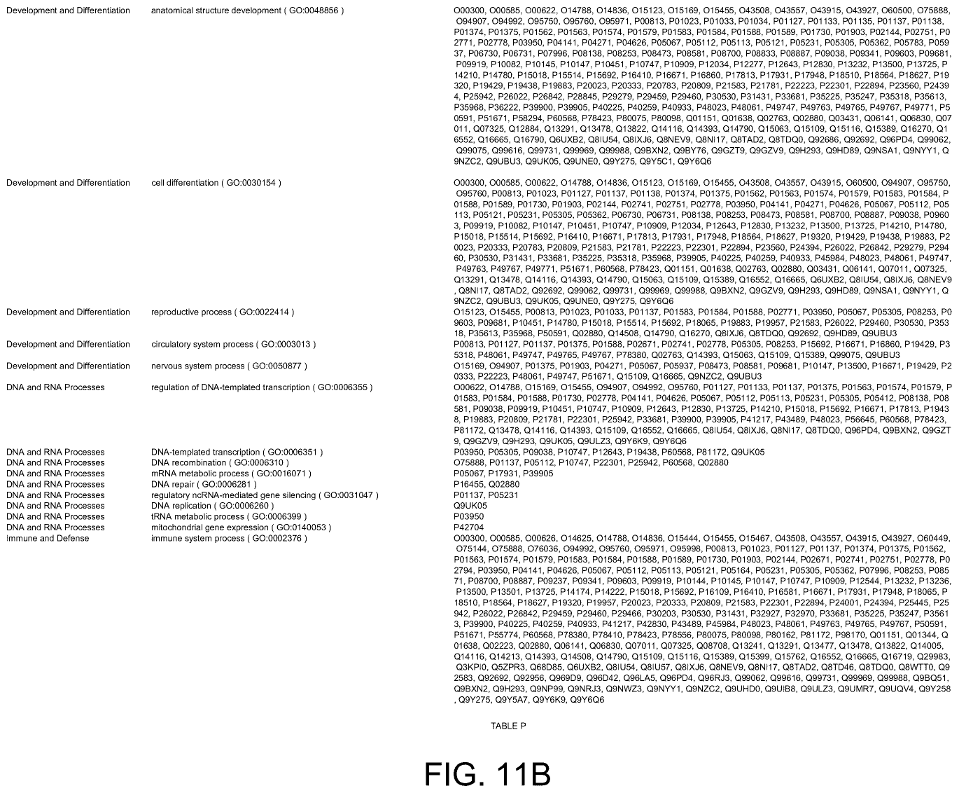

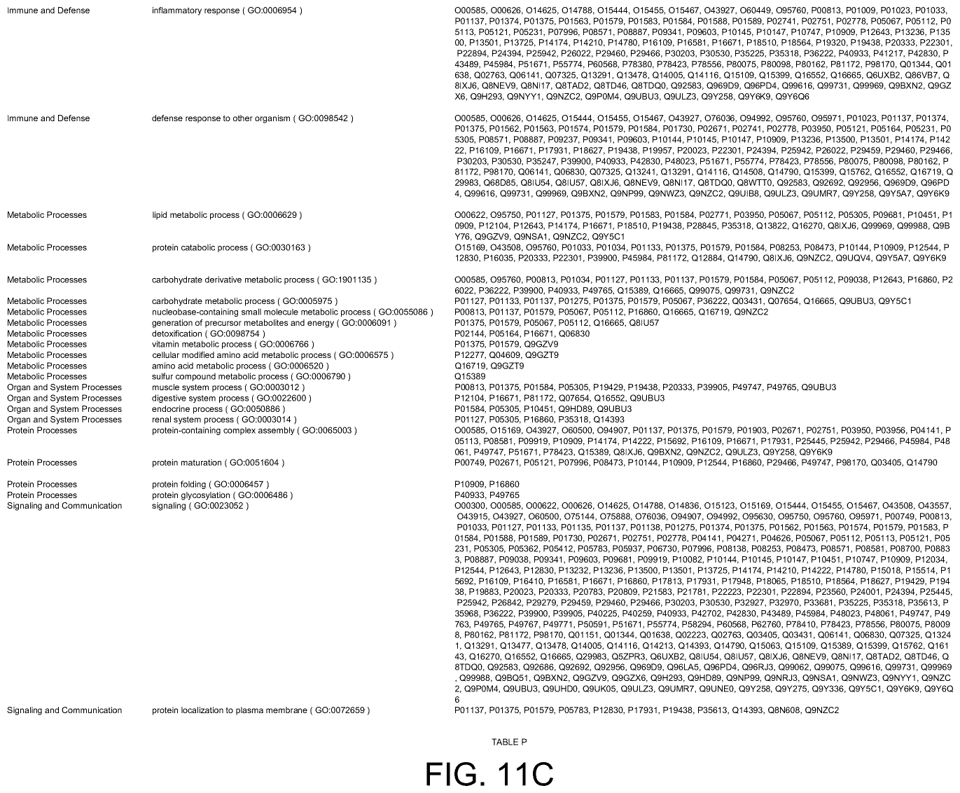

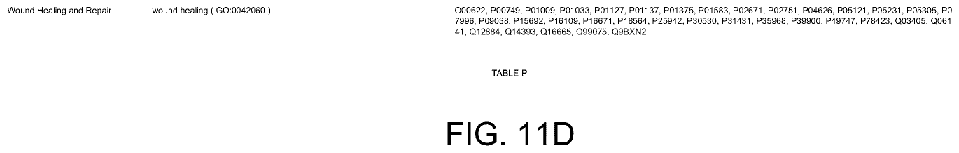

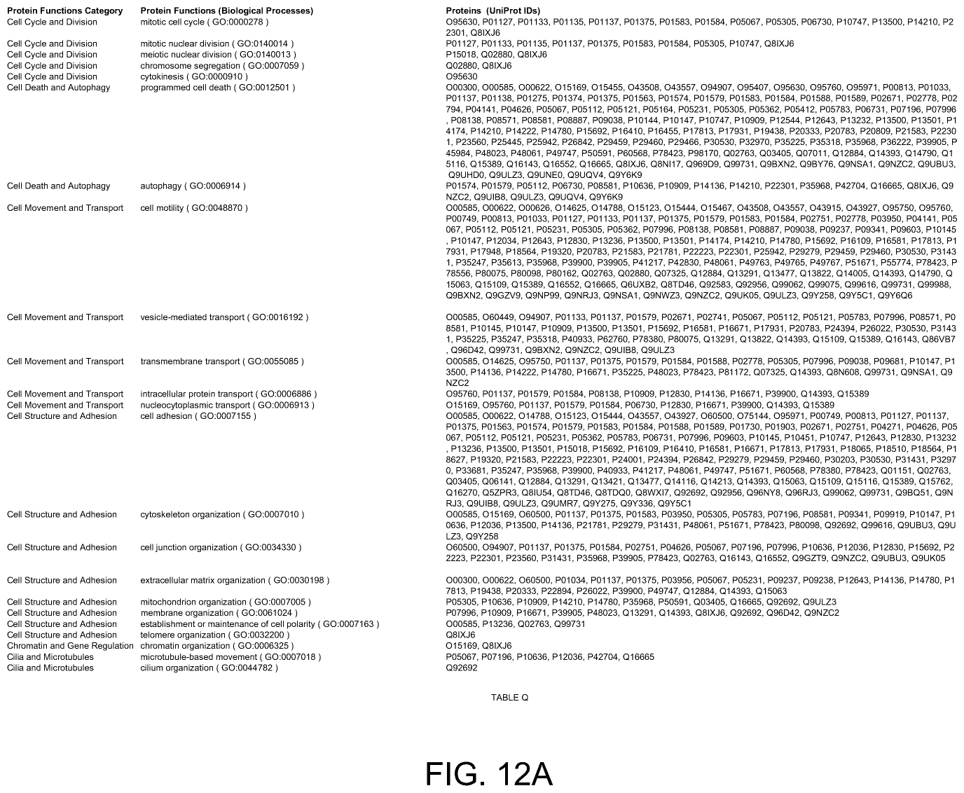

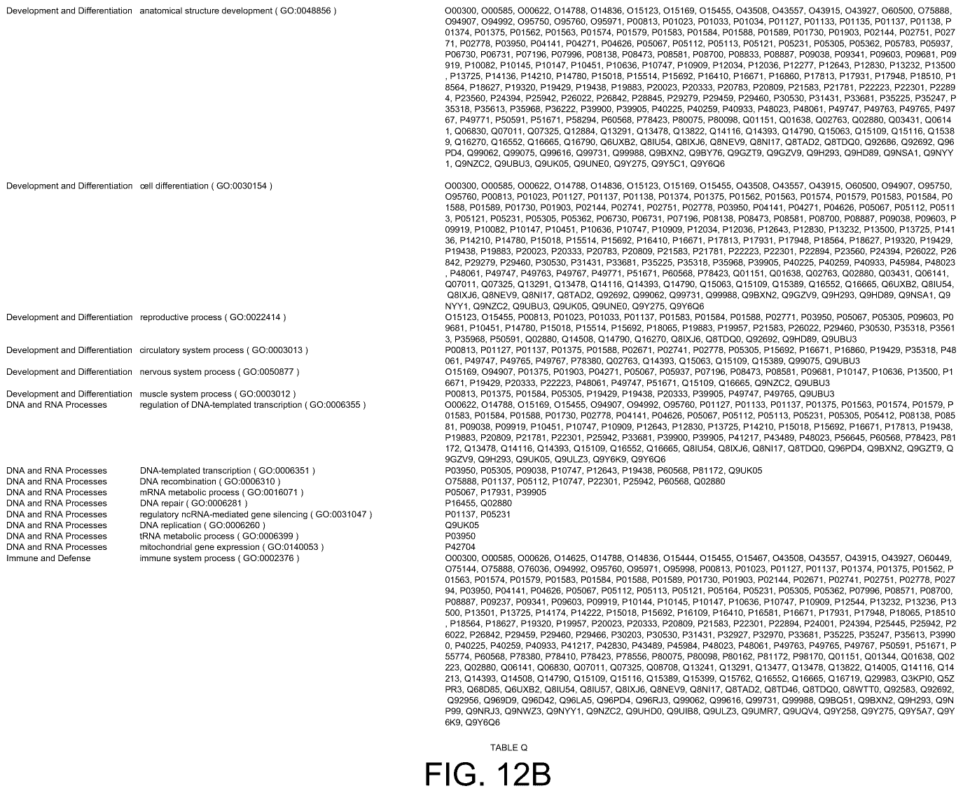

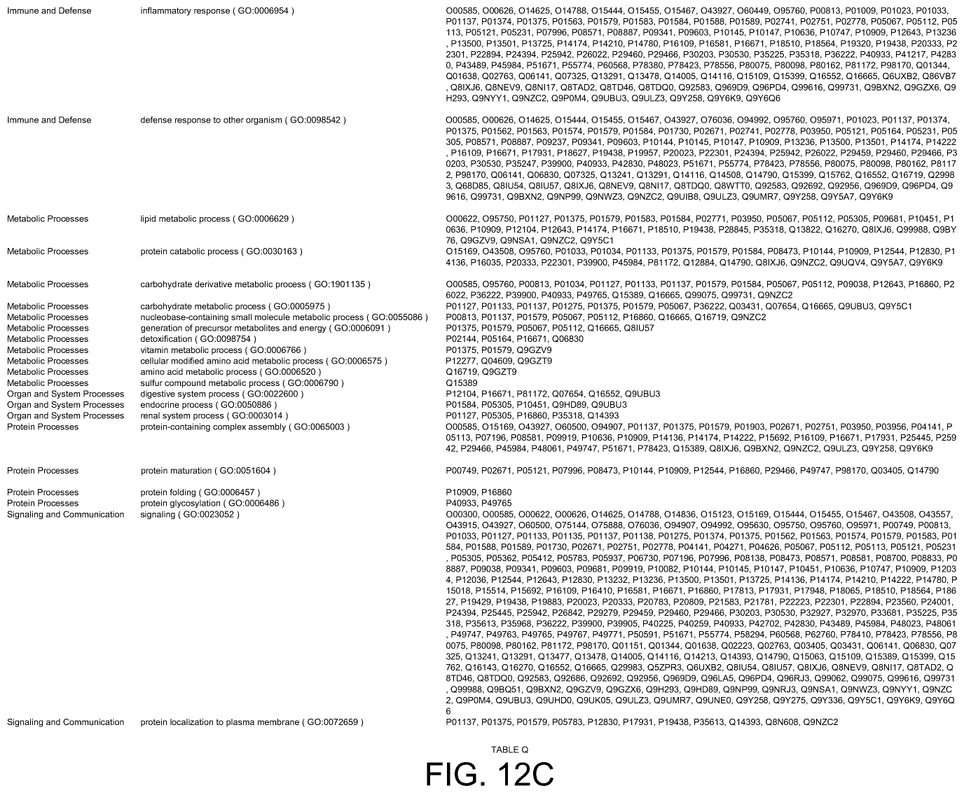

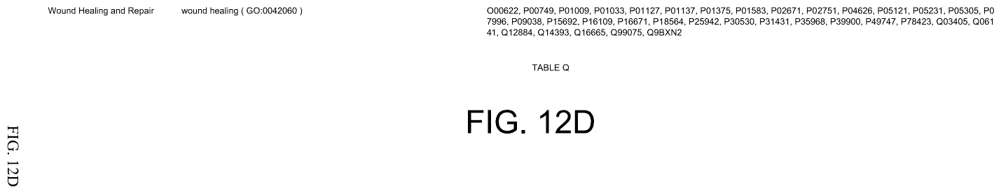

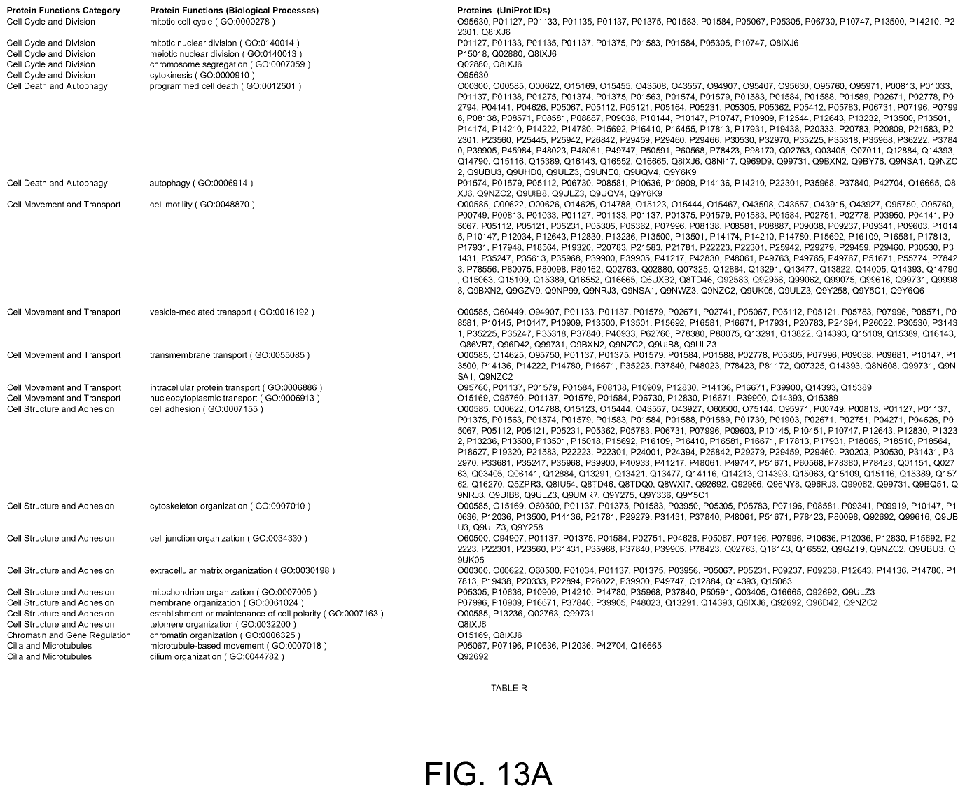

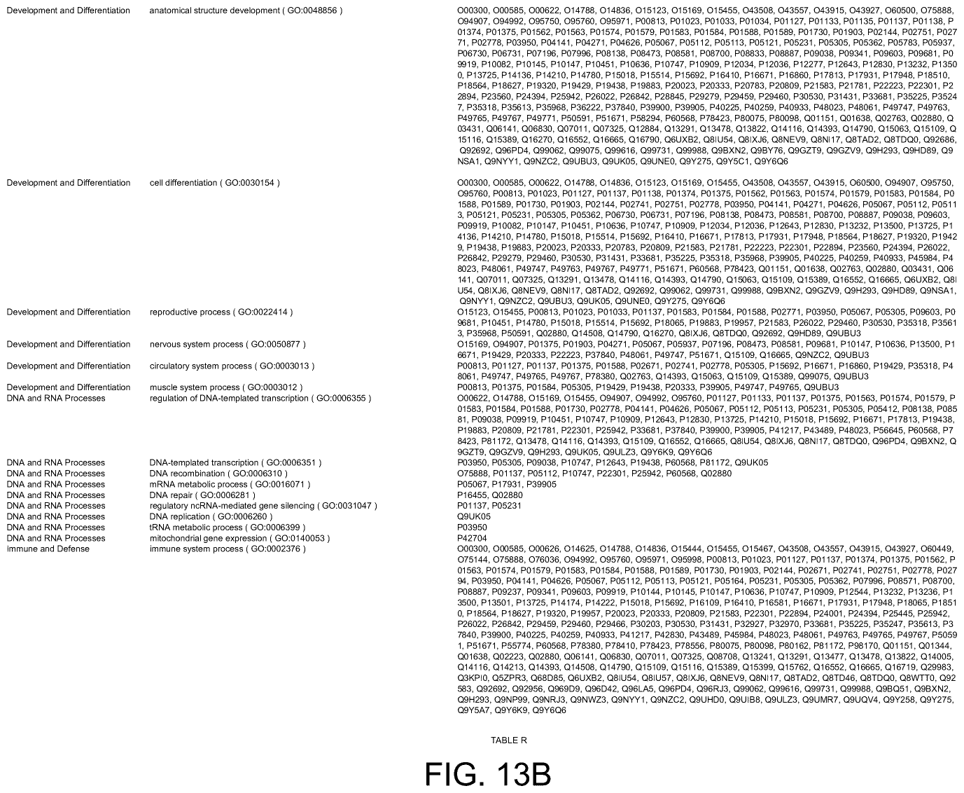

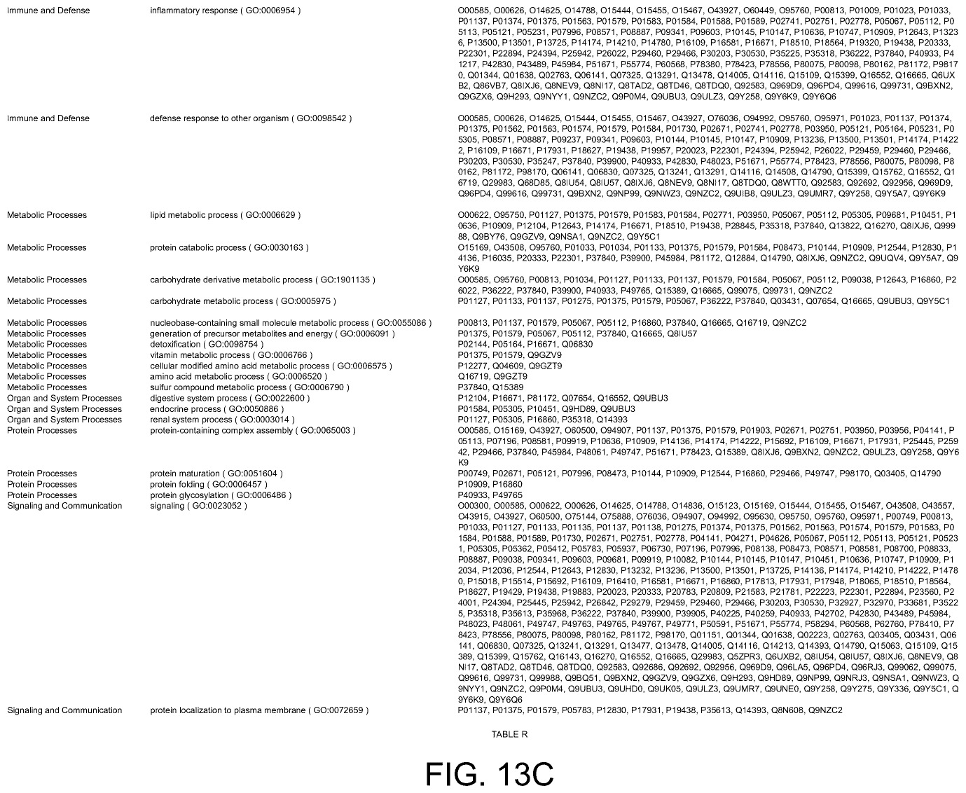

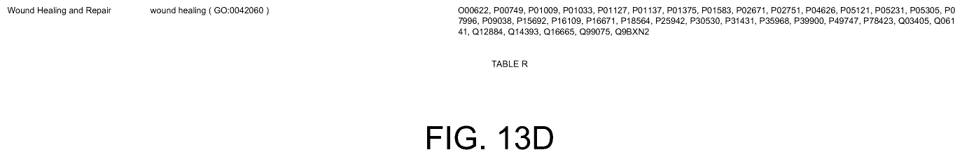

FIG. 6 illustrates an exemplary process for generating one or more prediction for one or more patient health characteristic, e.g., by utilizing one or more trained machine-learning model according to one or more embodiments discussed above. At step 605, patient medical information may be obtained. In an example, the patient medical information may include protein biomarker data.

At step 610, one or more machine-learning model may be selected, e.g., via the prediction system 120, from amongst a bank of trained machine-learning models, e.g., that are stored by the model system 115. In an example, one or more models may be selected at least in part based on a concordance of the one or more machine-learning model with one or more of the one or more patient health characteristic, patient data in the patient medical information, one or more time-frame for the prediction, or protein biomarkers included in the protein biomarker data. In an example, different machine learning models in the bank may have been one or more of trained using different stratifications of training patient data, trained to predict different patient health characteristics, trained or evaluated using different sets of protein biomarkers, or evaluated for different time-frames. Any of the different types of models, techniques for model generation, variable selection, parameter variation, or the like may have been used. In other words, the bank of models may include models having one or more varied parameters, such that one or more models may be selected based on particularities of not only the patient but also on the health characteristic(s) to be predicted and for which time-frames.

In an example, different machine-learning models in the bank may have been trained using different stratifications of training patient data. For instance, models configured to generate predictions for different characteristics may have been trained based a re-sampling of training patients having one or more particular characteristics. For example, different models may have been trained for different age brackets, genders, fitness levels, etc. In another example, different models may have been trained using different longitudinal segments of training data. For instance, one model might be trained using only patients for whom the training data includes 20 years of data, while another model might be trained using only patients for whom there is 10 years of data, or 10 or more years, etc.

In some embodiments, different machine-learning models in the bank may have been trained using different sets of protein biomarkers (e.g., different selections of proteins, groupings of proteins, categories of proteins, etc., or combinations thereof). Any suitable selection, such as those based on one or more of the other examples or embodiments disclosed herein, may be used. Different models in the bank may be evaluated for concordance between protein biomarkers and/or other patient characteristics obtained for the patient and the variables used in the different models. In an example, different protein assays may include data for different sets of proteins, and a model or models may be selected that have an optimal concordance with the data obtained for the patient.

In some embodiments, a plurality of machine-learning models may be selected from the bank. For example, each model of the plurality of selected models might be configured to generate a respective prediction for the one or more patient health characteristic at a different time-frame. For instance, a first model may generate a near term prediction within 5 years, another model may generate predictions for between 5 and 10 years, a further model might generate predictions for between 10 and 20 years, etc. As discussed in further detail below, multiple selected models may be used in combination, e.g., to generate a composite of their respective predictions. In some embodiments, different models in the bank may be evaluated for time-specific concordance and/or accuracy at different time frames, e.g., such that an optimal model for each time-frame may be selected from those available in the bank.

At step 615, the prediction system 120 may apply the one or more selected models to the obtained patient medical information in order to generate a prediction of the one or more patient health characteristics of interest. In an example, the prediction system 120 may output the prediction in conjunction with one or more baseline comparisons, e.g., a prediction for an average person overall, a prediction for a person having similar characteristics with the patient, etc. In another example, the prediction system 120 may combine predictions from a plurality of models to generate a composite prediction. For instance, the prediction system 120 may generate a plot of a patient's risk for a particular characteristic over time, in which different portions of the plot were sourced from different models.

FIG. 7 illustrates another exemplary process for generating one or more prediction for one or more patient health characteristic, e.g., by utilizing one or more trained machine-learning model according to one or more embodiments discussed above. At step 705, patient medical information may be obtained. The patient medical information may include protein biomarker data.

At step 710, a prediction of the patient health characteristic may be generated, e.g., by evaluating the patient medical information with a trained machine-learning model. In various embodiments, the machine-learning model may have been trained using a plurality of the proteins selected from one or more of the following categories: Cell Cycle and Division; Cell Death and Autophagy; Cell Movement and Transport; Cell Structure and Adhesion; Chromatin and Gene Regulation; Cilia and Microtubules; Development and Differentiation; DNA and RNA Processes; Immune and Defense; Metabolic Processes; Organ and System Processes; Protein Processes; Signaling and Communication; and Wound Healing and Repair. In various embodiments, the machine-learning model may have been trained using proteins selected from a listing or portions, a grouping of proteins, or a category of proteins from one or more of Tables A-R. In some embodiments, proteins from different categories and/or different groups are selected, e.g., to account for a larger scope of biological function.

In an example, a trained model configured to generate a prediction for longevity/all-cause mortality may have been trained using a plurality of the proteins from Table A. For instance, one or more of the pre-processing operations discussed above may have been used to identify a subset of the proteins listed in Table A. In another example, a plurality of proteins for training the model may have been selected based on one or more of the groups listed in Table G. In a further example, one or more proteins for training the model may have been selected based on one or more of groups and/or categories in Table M. Similar to the foregoing, Tables, B, H, and N pertain to heart function, Tables C, I, and O pertain to chronic kidney disease, Tables D, J, and P pertain to chronic obstructive pulmonary disease, Tables E, K, and Q pertain to dementia, and Tables F, L, and R pertain to Alzheimer's disease related plaque formation.

It should be understood that embodiments in this disclosure are exemplary only, and that other embodiments may include various combinations of features from other embodiments, as well as additional or fewer features.

In general, any process or operation discussed in this disclosure that is understood to be computer-implementable may be performed by one or more processors of a computer system, such any of the systems or devices in the environment, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices. One or more processors of a computer system may be included in a single computing device or distributed among a plurality of computing devices. A memory of the computer system may include the respective memory of each computing device of the plurality of computing devices. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, inventive aspects lie in less than all features of a single foregoing disclosed embodiment.

Furthermore, while some embodiments described herein include some but not other features included in other embodiments, combinations of features of different embodiments are meant to be within the scope of the invention, and form different embodiments, as would be understood by those skilled in the art.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted and operations may be interchanged. Steps may be added or deleted to techniques described within the scope of the present invention.

The above disclosed subject matter is to be considered illustrative, and not restrictive, and other implementations, which fall within the true spirit and scope of the present disclosure. Thus, to the maximum extent allowed by law, the scope of the present disclosure is to be determined by the broadest permissible interpretation, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for generating a prediction of one or more patient health characteristic, comprising:
obtaining patient medical information that includes protein data;
automatically selecting one or more machine-learning model from amongst a bank of trained machine-learning models based on a concordance of the one or more machine-learning model with one or more of the one or more patient health characteristic, patient data in the patient medical information, one or more time-frame for the prediction, or proteins included in the protein data, wherein different machine learning models in the bank were one or more of trained using different groupings of training patient data, trained to predict different patient health characteristics, trained or evaluated using different sets of proteins, or evaluated for different time-frames; and
generating the prediction of the one or more patient health characteristic by evaluating the patient medical information using the one or more selected machine learning model.

2. The computer-implemented method of claim 1, wherein:
the different machine learning models in the bank were trained using different groupings of training patient data; and
the grouping of the training patient data is based on one or more patient characteristic of patients associated with the training patient data.

3. The computer-implemented method of claim 1, wherein:
the different machine learning models in the bank were trained using different groupings of training patient data; and
the grouping of the training patient data is based on a longitudinal segmentation of the training patient data.

4. The computer-implemented method of claim 1, wherein:
a plurality of different machine-learning models are selected;
each model of the plurality of different machine-learning models is configured to generate a respective prediction for the one or more patient health characteristic at a different time-frame; and
generating, as the prediction, a composite of the respective predictions of the plurality of different machine-learning models.

5. The computer-implemented method of claim 4, wherein the model for each different time-frame is automatically selected based on evaluation of time-frame specific concordances or accuracies of the bank of trained machine-learning models.

6. The computer-implemented method of claim 1, wherein:
the different machine learning models in the bank were trained using different sets of proteins; and
the one or more trained machine-learning models are automatically selected based on concordance between the proteins included in the protein data and the different sets of proteins.

7. A computer-implemented method of generating a machine-learning model for predicting a patient health characteristic, the computer-implemented method comprising:
obtaining longitudinal medical information;
performing at least one pre-processing operation on the longitudinal medical information;
generating an ensemble of models that relate the longitudinal medical information to a patient health characteristic of interest;
applying the ensemble of models to at least a portion of the longitudinal medical information;
determining a sensitivity of variables of the longitudinal medical information by evaluating results from applying the ensemble of models to the longitudinal medical information;
identifying a subset of the variables based on the determined sensitivity;
training the machine-learning model using the subset of the variables of the longitudinal medical information

8. The computer-implemented method of claim 7, wherein the at least one preprocessing operation includes one or more of:
a down sampling operation;
a variable pruning operation; or
an interpolation operation.

9. The computer-implemented method of claim 8, wherein:
the at least one preprocessing operation includes a variable pruning operation; and
the variable pruning operation includes one or more of:
evaluating each of the variables for missing values and pruning any variable having a threshold number of missing values;
determining that a subset of the variables are one or more of correlated, deterministic of each other, and pruning at least one of the subset of variables having a low information inclusivity relative to others of the subset of variables.

10. The computer-implemented method of claim 7, wherein evaluating results from applying the ensemble of models to the longitudinal medical information includes a univariate analysis of the variables to determine the sensitivity.

11. The computer-implemented method of claim 10, wherein evaluating results from applying the ensemble of models to the longitudinal medical information includes a multivariate analysis of the variables to determine the sensitivity.

12. The computer-implemented method of claim 7, wherein the identifying of the subset of the variables is based having a high sensitivity relative to others of the variables.

13. The computer-implemented method of claim 7, wherein the longitudinal medical information include a plurality of proteins selected from one of Tables M through R.

14. The computer-implemented method of claim 7, wherein the subset of the variables include a plurality of proteins selected from one of Tables M through R.

15. The computer-implemented method of claim 7, wherein the longitudinal medical information includes proteins consisting of a plurality of proteins from protein groups selected from one of Tables M through R.

16. The computer-implemented method of claim 7, wherein the subset of the variables includes proteins consisting of a plurality of proteins from protein groups selected from one of Tables M through R.

17. The computer-implemented method of claim 7, wherein the longitudinal medical information includes proteins consisting of a plurality of proteins from protein categories selected from one of Tables M through R.

18. The computer-implemented method of claim 7, wherein the subset of the variables includes proteins consisting of a plurality of proteins from protein categories selected from one of Tables M through R.

19. A computer-implemented method for generating a prediction of one or more patient health characteristic, comprising:
obtaining patient medical information that includes protein data;
generating the prediction of the patient health characteristic by evaluating the patient medical information with a trained machine-learning model, wherein the machine-learning model has been trained using one or more of a plurality of the proteins, a plurality of protein groups, or a plurality of protein categories listed in Tables M through R.

20. The computer-implemented method of claim 19, wherein the one or more patient health characteristic includes one or more of longevity, heart function, kidney disease, obstructive pulmonary disease, or Alzheimer's disease-related plaque formation.
